(19) United States
(12) Patent Application Publication
(10) Pub. No.: US 2007/0082030 A1
(43) Pub. Date: Apr. 12, 2007

(54) Fiber satiety compositions

(76) Inventors: William R. Aimutis, Blaine, MN (US); Teresa Marie Paeschke, Minneapolis, MN (US); Norris Sun, Plymouth, MN (US); Scott Dale Johnson, Ephrata, PA (US); Alexander Patist, Minnetonka, MN (US); John F. Sweeney, St. Michael, MN (US); Terri Jo Vander Pol, Plymouth, MN (US)

Correspondence Address: FISH & RICHARDSON P.C., PO BOX 1022, MINNEAPOLIS, MN, 55440-1022, US

(21) Appl. No.: 11/246,938

(22) Filed: Oct. 7, 2005

(51) Int. Cl. A61K 47/00; A61K 33/42

(52) U.S. Cl. 424/439; 424/602

(57) ABSTRACT

Ingestible compositions that include one or more anionic soluble fibers are provided herein. An ingestible composition can be in solid or liquid form. In certain cases, a solid ingestible composition includes an extruded food product, such as a crispy, that includes one or more anionic soluble fibers. Liquid ingestible compositions that can include one or more alginate anionic soluble fibers and one or more pectin anionic soluble fibers are also provided. Methods for making ingestible compositions are provided, as well as methods for using the ingestible compositions, e.g., to induce satiety or to increase the viscosity of stomach contents.

### CROSS-REFERENCE TO RELATED APPLICATIONS

This case is related to U.S. patent application Ser No. ______, entitled “Fiber Satiety Compositions,” Attorney Docket number 10790-056001, filed concurrently herewith on Oct. 7, 2005.

This case is also related to U.S. application Ser. No. ______, entitled “Compositions And Methods For Reducing Food Intake And Controlling Weight,” Attorney Docket number MSP5038; Ser. No. ______, entitled “Methods for Reducing Calorie Intake,” Attorney Docket number MSP5039; Ser. No. ______, entitled “Compositions and Methods for Inducing Satiety And Reducing Caloric Intake,” Attorney Docket number MSP5040; Ser. No. ______, entitled “Methods For Achieving And Maintaining Weight Loss,” Attorney Docket number MSP5041; Ser. No. ______, entitled “Methods For Reducing Weight,” Attorney Docket number MSP5042; Ser. No. ______, entitled “Compositions and Methods for Reducing Food Intake and Controlling Weight,” Attorney Docket number MSP5043; Ser. No. ______, entitled “Compositions and Methods for Reducing Food Intake and Controlling Weight,” Attorney Docket number MSP5044; Ser. No. ______, entitled “Compositions And Methods For Reducing Food Intake And Controlling Weight,” Attorney Docket number MSP5045; Ser. No. ______, entitled “Methods For Inducing Satiety, Reducing Food Intake And Reducing Weight,” Attorney Docket number MSP5046; and Ser. No. ______, entitled “Compositions and Methods for Reducing Food Intake and Controlling Weight,” Attorney Docket number MSP5047; each of which was filed concurrently herewith on Oct. 7, 2005.

### TECHNICAL FIELD

Provided herein are ingestible compositions that include one or more anionic soluble fibers, methods for making ingestible compositions, and methods of using ingestible compositions to facilitate satiety and/or satiation and to increase the viscosity of stomach and/or small intestinal contents. Also provided are fiber blends, e.g., for preparing the ingestible compositions.

### BACKGROUND

Diabetes and obesity are two of the most significant and costly health problems facing the United States. Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet, however, often due to the negative organoleptic characteristics, such as sliminess, excessive viscosity, and poor flavor, that are associated with food products that include soluble fiber.

### SUMMARY

Provided herein are fiber blends, ingestible compositions, materials and methods for making the same, and materials and methods for using the same. A fiber blend can include one or more anionic soluble fibers. For example, a fiber blend can include alginate and pectin anionic soluble fibers. An alginate for use in the blend can include both intermediate and low molecular weight distribution range forms of alginate. A pectin can be a high-methoxy pectin. Fiber blends can be used in the preparation of both solid and liquid ingestible compositions.

A solid ingestible composition includes one or more anionic soluble fibers. In some cases, a solid ingestible composition includes two or more anionic soluble fibers. Anionic soluble fibers can be treated so that they are in a form that aids hydration and reduces sliminess upon consumption. For example, a solid ingestible composition can include an extruded food product, such as a crispy, where the extruded food product includes the one or more anionic soluble fibers. Inclusion of an extruded crispy in a solid ingestible composition can reduce sliminess in the mouth while also aiding in hydration and gelation of the soluble fibers (e.g., with available cations) once the fibers are in the acidic environment of the stomach. Such hydration and gelation can lead to increased viscosity of stomach contents, possibly inducing satiety and/or increasing the time period of satiation.

In some cases, a solid ingestible composition that includes one or more anionic soluble fibers is an extruded food product. For example, an extruded bar or cookie, such as an extruded granola bar or cookie, that includes one or more anionic soluble fibers is provided herein. In some cases, a solid ingestible composition that includes one or more anionic soluble fibers includes an extruded food product, e.g., a granola bar that includes extruded crispies. In some cases, an ingestible composition can include a layer that also includes one or more anionic soluble fibers, e.g., a gel layer that includes one or more anionic soluble fibers. In some cases, an ingestible composition can include a layer that includes a source of one or more cations. A gel layer can be one that is traditionally acceptable to the consumer such as, without limitation, a jelly, jam, pudding, fruit sauce, yogurt layer, etc.

Liquid ingestible compositions that include one or more anionic soluble fibers are also provided. In some cases, a liquid ingestible composition includes two or more anionic soluble fibers, e.g., alginate anionic soluble fibers and pectin anionic soluble fibers. Without being bound by theory, it is believed that such beverages can exhibit improved gel strength in the stomach, with a reduced amount of precipitation of the alginate and pectin than expected at the lower pHs of the stomach. An alginate anionic soluble fiber can be a high guluronic acid alginate and can include a higher than 1:1 ratio of guluronic acids relative to mannuronic acids. A pectin anionic soluble fiber can be a high-methoxy pectin (e.g., greater than about 50% esterified carboxylates). In certain cases and in certain ratios relative to the alginate, it is believed that pectin can provide a protective stabilizing effect on the alginate, e.g., to prevent precipitation in the acidic stomach.

Materials and methods for sequestering or protecting cations, including calcium, in the described ingestible compositions are also provided herein. While not being bound by theory, it is believed that inclusion of a source of a cation in an ingestible composition, such as a protected source of calcium, can induce and/or stabilize an anionic fiber gel in the stomach or can provide stabilization as a gel moves into the more alkaline environment of the small intestine. The materials and methods for cation sequestration and protection can promote the induction of satiety and/or increase the time period of satiation after a meal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph demonstrating the molecular weight distribution of alginate polymers using SEC with RI and MALLS detection.

FIG. 2 is a graph showing the gel strength for a variety of different alginates.

FIG. 3 is a graph showing the gel strength for various liquid ingestible compositions provided herein.

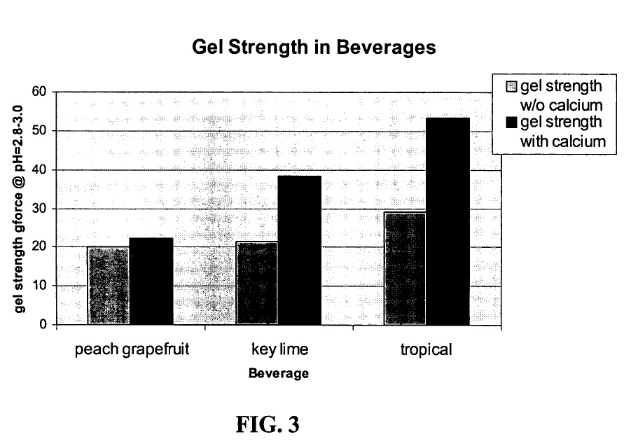

FIG. 4 is a graph showing viscosity measurements for pig stomach digesta after consumption of an anionic soluble fiber-containing beverage and after consumption of the same beverage followed by a source of calcium.

FIG. 5 is a graph showing viscosity measurements for pig duodenal fluid after consumption of the same beverages as in FIG. 4.

### DETAILED DESCRIPTION

Materials and methods for the preparation of fiber blends and solid and liquid ingestible compositions, including snack bars, cookies, brownies, breads, bagels, cereals, pastas, crispies, beverages, and smoothies, are provided herein. Certain of the described ingestible compositions can exhibit reduced sliminess and toothpack and improved palatability relative to other ingestible compositions that incorporate soluble fibers. While not being bound by any theory, the ingestible compositions can induce satiety and/or an increased time period of satiation, thus aiding in weight loss. Ingestion of the ingestible compositions could also aid in glycemic control and decreased blood cholesterol levels.

Ingestible Compositions and Articles of Manufacture

An ingestible composition can be in solid (e.g., bar, cookie, bread, etc.) form or liquid form (beverage, smoothie, shake, etc.). An ingestible composition can be provided in any packaging, such as enclosed in a wrapper or included in a container. An ingestible composition can be included in an article of manufacture. An article of manufacture that includes an ingestible composition described herein can include auxiliary items such as straws, napkins, labels, packaging, utensils, etc. An article of manufacture can include a source of one or more cations. For example, a source of one or more cations can be provided as a liquid, e.g., as a beverage to be consumed before, during, or after ingestion of the ingestible composition. In other cases, a source of one or more cations can be provided in a solid or gel form. For example, a source of one or more cations can be provided in e.g., a jelly, jam, dip, or pudding, to be eaten before, during, or after ingestion of the ingestible composition. Thus, in some embodiments, an article of manufacture that includes a cookie or bar solid ingestible composition can also include a dip comprising a source of one or more cations, e.g., into which to dip the cookie or bar solid ingestible composition.

Solid Ingestible Compositions

Solid ingestible compositions, such as, without limitation, bars, breads, muffins, cookies, brownies, cereals, chips, nuggets, snack foods, bagels, chews, crispies, pastas, and nougats, are provided herein. A solid ingestible composition can include one or more anionic soluble fibers. In some cases, a solid ingestible composition can include two or more anionic soluble fibers. Solid ingestible compositions can provide from about 0.5 g to about 10 g total anionic soluble fiber per serving, e.g., about 0.5 g to about 5 g, about 1 g to about 6 g, about 3 g to about 7 g, about 5 g to about 9 g, or about 4 g to about 6 g. As used herein, a recitation of a range of values is merely intended to serve as a shorthand method of referring individually to each separate value falling within the range, and each separate value is incorporated into the specification as if it were individually recited herein. For example, in some cases, about 1 g, about 2 g, about 3 g, about 4 g, about 5 g, about 6 g, about 7 g, about 8 g, or about 9 g of anionic soluble fiber per serving can be provided. As used herein, unless indicated otherwise, a serving is considered to be the “reference amount customarily consumed” or RACC.

A solid ingestible composition can include one or more anionic soluble fibers at a total weight percent of the ingestible composition of about 4% to about 50%. For example, a solid ingestible composition can include one or more anionic soluble fibers from about 4% to about 10% by weight; or about 5% to about 15% by weight; or about 10% to about 20% by weight; or about 20% to about 30% by weight; or about 30% to about 40% by weight; or about 40% to about 50% by weight.

One or more anionic soluble fibers can be present in a solid ingestible composition in either a processed or unprocessed form (e.g., relative to the soluble fiber after extraction and purification from its source). For example, anionic soluble fibers can be processed, without limitation, by extrusion (cold or hot, high pressure or low pressure), spray-drying, roll-drying, dry-blending, roll-blending, and freeze-drying. One or more anionic soluble fibers can be present in a solid ingestible composition in one or more processed forms (e.g., an extruded food product, such as a crispy as described below) or in an unprocessed forms (e.g., a formed dough or composition), or both. For example, a snack bar solid ingestible composition can include one or more anionic soluble anionic fibers present as an extruded food product (e.g., a crispy), one or more anionic soluble fibers in an unextruded form (e.g., a formed bar), or both (e.g., a formed bar that includes crispies). A snack chip solid ingestible composition can include one or more soluble anionic fibers in extruded form or in spray-dried form, or both, e.g., an extruded anionic soluble fiber-containing chip having one or more anionic soluble fibers spray-dried on the chip. A cookie solid ingestible composition can include one or more soluble anionic fibers in an unprocessed form (e.g., a formed cookie) or in a processed (e.g., extruded) form, or both (e.g., a formed cookie that includes crispies).

A solid ingestible composition can include optional ingredients such as frostings, coatings, drizzles, chips, chunks, swirls, or layers. Such optional ingredients can be a source of one or more cations, as described further herein. Such optional ingredients can be a source of one or more anionic soluble fibers, e.g., pectin in a jelly layer.

A solid ingestible composition can include an extruded food product. An extruded food product can be cold- or hot-extruded under high or low pressure and can assume any type of extruded shape, including without limitation, a bar (e.g., a nutritional bar or meal replacement bar), cookie, bagel, crispy, puff, curl, crunch, ball, flake, square, nugget, and chip. In some cases, an extruded food product is in bar shape, such as a snack bar, granola, nutritional bar, or meal replacement bar. In some cases, an extruded food product is in cookie shape. In other cases, an extruded food product is in a shape such as a crispy, puff, flake, curl, ball, crunch, nugget, chip, square, chip, pasta, or nugget. Such extruded food products can be eaten as is (e.g., cookies, bars, chips, crispies as cereal) or can be incorporated into a solid ingestible composition, e.g., crispies incorporated into snack bars.

An extruded food product, such as, without limitation, a crispy, puff, flake, curl, nugget, or ball, can have a bulk density in the range from about 0.08 to about 0.25 gram/cubic centimeter, e.g., from about 0.1 to about 0.22; from about 0.1 to about 0.19; or from about 0.1 to about 0.15 gram/cubic centimeter.

An extruded food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, an extruded bar, cookie, or chip can be about 80% to about 100% by weight of an ingestible composition, such as a snack bar ingestible composition or a cookie ingestible composition, respectively. Thus, in some embodiments, a cold-extruded cookie or bar can be from about 80% to about 100% by weight of an ingestible composition, where the remainder of the weight derives from frostings, toppings, etc.

Alternatively, an ingestible composition can include about 30% to about 55% by weight of an extruded food product, e.g., about 32%, 33%, 34%, 35%, 36%, 37%, 38%, 39%, 40%, 42%, 45%, 48%, 50%, 52%, or 54% by weight. For example, an ingestible snack bar composition can include about 32% to about 46% by weight of extruded crispies. Such a crispy can be hot-extruded under high-pressure.

The amount of the one or more anionic soluble fibers included in a solid ingestible composition can vary, and will depend on the type of solid ingestible composition and the type of anionic soluble fiber. As indicated previously, typically a solid ingestible composition will include from about 0.5 g to about 10 g total anionic soluble fiber per serving, or any value therebetween.

In certain cases, an extruded food product can include an anionic soluble fiber at from about 22% to about 40% by weight of the extruded product, or any value therebetween, e.g., from about 29% to about 33%, about 30% to about 32%, or about 22% to about 27% by weight. For example, an extruded crispy can include about 31.5% of total alginate anionic soluble fiber, such as about 31.5% of two or more alginate forms, or about 31.5% total of two or more alginate forms and a pectin form. In other cases, an extruded food product can include an anionic soluble fiber in an amount of from about 4% to about 15%, such as when only gellan is used. In yet other cases, an extruded food product can include an anionic soluble fiber at a total amount of from about 18% to about 25% by weight, for example, when combinations of gellan and alginate or gellan and pectin are used.

An anionic soluble fiber for inclusion in a solid ingestible composition, liquid ingestible composition as described below, or fiber blend as described below, can be selected from alginate, pectin, carrageenan, polygeenan, and gellan. As used herein, unless indicated otherwise, the term “alginate,” “pectin,” “carrageenan,” “polygeenan,” or “gellan” refers to all forms (e.g., protonated or salt forms, such as sodium, potassium, calcium, magnesium, and ammonium salt forms and forms having varying average molecular weight distribution ranges) of the anionic soluble fiber type. Thus, in certain cases, two or more anionic soluble fibers types are included, such as alginate and pectin, alginate and gellan, or pectin and gellan. In other cases, only one type of anionic soluble fiber is used, such as only alginate, only pectin, only carrageenan, or only gellan. When an anionic soluble fiber type is used (e.g., alginate), in certain cases, more than one form of that type can be used, e.g., an intermediate molecular weight alginate and a low molecular weight alginate can be used, as described more fully below.

An alginate can be a high guluronic acid alginate. For example, in certain cases, an alginate can exhibit a higher than 1:1 ratio of guluronic to mannuronic acids, such as in the range from about 1.2:1 to about 3:1, e.g., about 1.3:1, about 1.4:1, about 1.5:1, about 1.6:1, about 1.7:1, about 1.8:1, about 2:1, about 2.2:1, about 2:5:1, about 2:8:1, and about 2:9:1. Examples of high guluronic alginates (e.g., having a higher than 1:1 g:m ratios) include Manugel LBA, Manugel GHB, and Manugel DBP, which each have a g:m ratio of about 1.5:1. Manugel DMB and Protanal LF5/60 can also be used.

In other cases, an alginate can exhibit a ratio of guluronic to mannuronic acids (g:m ratio) of less than 1:1, e.g., 0.8:1 to about 0.4:1, such as about 0.5:1, about 0.6:1, or about 0.7:1. Keltone LV and Keltone HV are examples of high-mannuronic acids (e.g., having a g:m ratio of less than 1:1). Methods for measuring the ratio of guluronic acids to mannuronic acids are known by those having ordinary skill in the art.

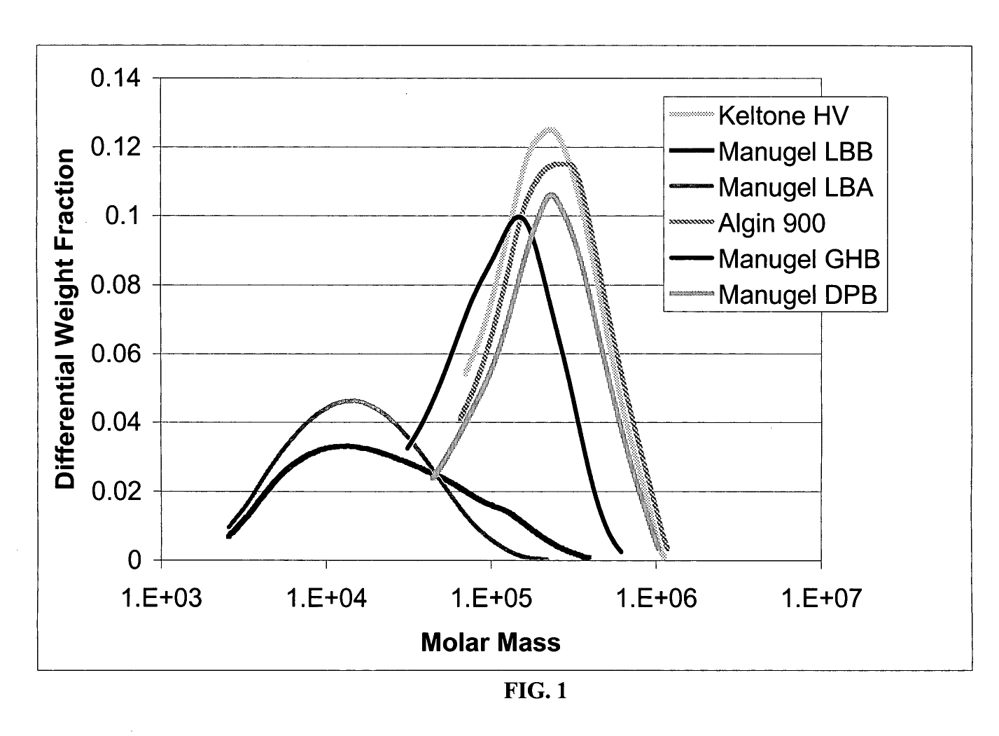

An alginate can exhibit any number average molecular weight distribution range, such as a high molecular weight distribution range (about 5×10⁴ to about 1×10⁶ molar mass; examples include Manugel DPB, Keltone HV, and TIC 900 Alginate); an intermediate molecular weight distribution range (about 1×10⁴ to about 2×10⁵ molar mass; examples include Manugel GHB); or a low molecular weight distribution range (1×10² to about 1×10⁵ molar mass; examples include Manugel LBA and Manugel LBB). Number average molecular weights can be determined by those having ordinary skill in the art, e.g., using size exclusion chromatography (SEC) combined with refractive index (RI) and multi-angle laser light scattering (MALLS); see FIG. 1.

In certain embodiments, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and intermediate molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CP Kelco USPL200. A pectin can exhibit any number average molecular weight range, including a low molecular weight distribution range (about 1×10³ to about 8×10⁵ molar mass, e.g., CP Kelco USPL200), an intermediate molecular weight distribution range (about 1×10⁴ to about 1×10⁶, e.g., ISP HM70LV), or high molecular weight distribution range (about 1×10⁴ to about 1×10⁶, e.g., TIC HM Pectin). In certain cases, a high-methoxy pectin can be obtained from pulp, e.g., as a by-product of orange juice or other fruit processing.

A gellan anionic soluble fiber can also be used. Gellan fibers form strong gels at lower concentrations than alginates and/or pectins, and can cross-link with mono- and divalent cations. For example, gellan can form gels with sodium, potassium, magnesium, and calcium. Gellans for use in the invention include Kelcogel, available commercially from CP Kelco.

Anionic soluble fibers such as alginate, pectin, carrageenan, and gellan are commercially available, e.g., from ISP (Wayne, NJ), TIC Gums, and CP Kelco.

Fiber blends as described further herein can also be used in the preparation of a solid ingestible composition or extruded food product. For example, a fiber blend can be used as a source of one or more soluble anionic fibers. A useful fiber blend can include an alginate soluble anionic fiber and a pectin soluble anionic fiber. An alginate soluble anionic fiber in a blend can be a mixture of two or more alginate forms, e.g., an intermediate and low molecular weight alginate; a high molecular weight and low molecular weight alginate; two intermediate molecular weight alginates; or two low molecular weight alginates.

In addition to one or more anionic soluble fibers, a solid ingestible composition or extruded food product can include ingredients that may be treated in a similar manner as the one or more anionic soluble fibers. For example, an ingredient can be co-processed with an anionic soluble fiber, such as co-extruded, co-roll-blended, or co-spray-dried with an anionic soluble fiber.

Additional ingredients can be hydrophilic in nature, such as starch, protein, maltodextrin, and inulin. These ingredients may help disperse the anionic soluble fibers, reduce sliminess, and aid in hydration after digestion. Other additional ingredients can be insoluble in water (e.g., cocoa solids, corn fiber); can be fat soluble (vegetable oil); or can be flavor modifiers, such as sucralose. For example, an extruded food product can include from about 5 to about 80% of a cereal ingredient, such as about 40% to about 68% of a cereal ingredient. A cereal ingredient can be rice, corn, wheat, sorghum, oat, flax or barley grains, flours, or meals. For example, an extruded food product such as a crispy can include about 40% to about 50%, about 50% to about 58%, about 52% to about 57%, or about 52%, 53%, 54%, 55%, 56%, or 56.5% by weight of a cereal ingredient. In one embodiment, about 56.5% of rice flour is included.

A solid ingestible composition can also include a protein source. A protein source can be included in the composition or in an extruded food product incorporated into a solid ingestible composition. For example, an extruded food product can include a protein source at about 2% to about 20% by weight, such as about 3% to about 8%, about 3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

An ingestible composition or extruded food product can include one or more of the following: cocoa, insoluble fibers, insoluble cellulosic material (e.g., microcrystalline cellulose, such as Avicel (FMC, Philadelphia, Pa.) or Solka Floc (International Fiber Corporation, North Tonawanda, N.Y.)), and oils or fats derived from animal or vegetable sources, e.g., soybean oil, canola oil, corn oil, safflower oil, sunflower oil, palm, palm kernel, etc. For example, an extruded food product can include about 3% to about 10% (e.g., about 3% to about 6%, about 4% to about 6%, about 5%, about 6%, about 7%, or about 4% to about 8%) by weight of such an added ingredient.

Crispies

An extruded food product, e.g., for inclusion in a solid ingestible composition, can be a crispy. For example, crispies that include one or more alginates and/or pectins in a total amount of about 30% to about 35% by weight can be included in a snack bar in an amount of about 32% to about 45% by weight of the snack bar. Crispies can be prepared using a fiber blend as described herein. Crispies can also include, among other things, about 52% to about 58% by weight of one or more of a rice flour, corn meal, and/or corn cone; and about 2% to about 10% of a protein isolate. Crispies can be prepared using methods known to those having ordinary skill in the art, including hot extrusion techniques.

For example, in one embodiment, to produce a batch of crispies, ingredients may be dry blended in a small ribbon blender. The resulting dry blend can be transferred using a feeder, e.g., a K-Tron loss-in-weight feeder, into the hopper of an extruder, e.g., a Buhler Twin Screw Extruder configured with at least one heating unit, e.g., two Mokon barrel-heating units. Water can be added as steam to the dry blend, using a barrel injection system. A second liquid could also be introduced at variable rates using another injector. The blend is then mixed and cooked in the extruder. Hot pressured product stream is then forced through a die for expansion, cut, and then conveyed by vacuum or mechanical conveying to a fluid bed drier, e.g., Buhler fluid bed drier, and dried to the desired moisture content. The fluid bed drier can dry about 50 to about 100 kg/hour at temperatures from about 20 to about 110° C.

Methods for Treatment or Processing ofAnionic Soluble Fibers

One or more anionic soluble fibers may be treated before, during, or after incorporation into an ingestible composition. For example, one or more anionic soluble fibers can be treated, such as extruded, roll-dried, freeze-dried, dry blended, roll-blended, or spray-dried. Extrusion can be cold or hot extrusion, and/or under high or low pressure. A variety of extruded shapes of food products can be prepared by methods known to those having ordinary skill in the art. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process, e.g., where a mixture is extruded through a die and subjected to a cutter to portion the mixture. Extruders are commercially available, e.g., from Buhler, Germany.

Other processing methods are known to those having ordinary skill in the art.

Liquid Ingestible Compositions

Liquid ingestible compositions, such as beverages, shakes, and smoothies, are also provided herein. Liquid ingestible compositions can be useful for, among other things, aiding in weight loss programs, e.g., as meal replacement beverages or diet drinks. Liquid ingestible compositions can provide from about 0.25 g to about 10 g of anionic soluble fiber per serving, or any value or range therebetween (e.g., about 2 g to about 5.6 g; about 0.5 g to about 3 g per serving; about 1 g to about 2 g per serving). For example, in certain cases, about 0.25 g, 0.5 g, 0.75 g, 1 g, 2 g, 3 g, 4 g, 5 g, 6 g, 7 g, 8 g, or 9 g of one or more anionic soluble fibers are provided per serving. In certain cases, from 1 to 2 g of anionic soluble fibers are provided per serving, e.g., 1.1 g, 1.2 g, 1.3 g, 1.4 g, 1.5 g, 1.6 g, 1.7 g, 1.8 g, or 1.9 g of anionic soluble fiber per serving.

A liquid ingestible composition includes an alginate anionic soluble fiber and/or a pectin anionic soluble fiber. In certain cases, an alginate anionic soluble fiber and a pectin anionic soluble fiber are used. A fiber blend as described herein can be used to provide the alginate anionic soluble fiber and/or the pectin anionic soluble fiber. An alginate and a pectin can be in any form, as described previously. For example, an alginate can be a high, intermediate, or low molecular weight distribution range alginate, and a pectin can be a high-methoxy pectin. Also as indicated previously, two or more alginate forms can be used, such as a high molecular weight and a low molecular weight distribution range alginate, or two high molecular weight distribution range alginates, or two low molecular weight distribution range alginates, or a low and an intermediate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an anionic soluble fiber can be used, e.g., to reduce the sodium content of an ingestible composition.

A liquid ingestible composition includes alginate and/or pectin in a total amount of about 1.0% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. In some embodiments, about 1.7% fiber by weight of a liquid ingestible composition is targeted.

In some cases, a liquid ingestible composition includes only alginate as a soluble anionic fiber. In other cases, alginate and pectin are used. When both alginate and pectin are used, the two soluble anionic fibers can be provided as a fiber blend of the two fibers in the appropriate ratios. A ratio of alginate to pectin (e.g., total alginate to total pectin) in a liquid ingestible composition can range from about 8:1 to about 1:8, and any ratio therebetween (e.g., alginate:pectin can be in a ratio of about 1:1, 1.2:1, 1.3:1, 1.4:1, 1.5:1, 1.6:1, 1.62:1, 1.7:1, 1.8:1, 1.9:1, 2:1, 3:1, 4:1, 5:1, 5.3:1, 5.6:1, 5.7:1, 5.8:1, 5.9:1, 6:1, 6.1:1, 6.5:1, 7:1, 7.5:1, 7.8:1, 2:3, 1:4, or 0.88:1). In cases where alginate and pectin are in a ratio of about 0.5:1 to about 2:1, it is believed that pectin and alginate electrostatically interact with one another to gel in the absence of cations; thus, while not being bound by theory, it may be useful to delay the introduction of cations (see methods below) until after such gel formation. In other cases, where the ratio of alginate to pectin is in the range from about 3:1 to about 8:1, it may be useful to include a cation source such as a calcium source (e.g., to crosslink the excess alginate) to aid gel formation in the stomach. In these cases, the inventors believe, while not being bound by any theory, that the lower amount of pectin protects the alginate from precipitating as alginic acid at the low pH values of the stomach environment, while the cation source cross-links and stabilizes the gels formed.

An liquid ingestible composition can have a pH from about 3.9 to about 7.5. In certain cases, a liquid ingestible composition can have a pH from about 3.9 to about 4.5, e.g., about 4.0 to about 4.3, or about 4.1 to about 4.2. At these pHs, it is believed that the liquid ingestible compositions are above the pKas of the alginate and pectin acidic subunits, minimizing precipitation, separation, and viscosity of the solutions. In some cases, malic, phosphoric, and citric acids can be used to acidify the compositions. In certain cases, a liquid ingestible composition can have a pH of from about 4.5 to about 7.5. Such liquid ingestible compositions can use pH buffers known to those having ordinary skill in the art.

Sweeteners for use in a liquid ingestible composition can vary according to the use of the composition. For diet beverages, low glycemic sweeteners and/or high intensity sweeteners may be preferred, such as polyols, trehalose, isomaltulose, and sucralose. Sucralose and/or other high intensity sweeteners such as aspartame, neotame, acesulfame K, etc. can be used alone in certain formulations. The choice of sweetener will impact the overall caloric content of a liquid ingestible composition. In certain cases, a liquid ingestible composition can be targeted to have about 40 calories/12 oz serving.

A liquid ingestible composition can demonstrate gel strengths of about 20 to about 250 grams Force (e.g., about 60 to about 240, about 150 to about 240, about 20 to 30, about 20 to about 55, about 50 to 200; about 100 to 200; and about 175 to 240), as measured in a gel strength assay (see Examples, below). Gel strengths can be measured in the presence and absence of a cation source, such as a calcium source. For example, a gel screening technique can be used to assess the gel strength of anionic soluble fibers and fiber blends (in the presence and absence of cations) and without shear and enzymes. The method includes lowering the pH of a solution containing one or more soluble fibers, and adding a source of cations (e.g., calcium). In some embodiments, glucono delta lactone (GDL) can be used as an acidulant, as it dissociates to lower the pH gradually. Dicalcium phosphate can be used a slowly soluble calcium source, which becomes more soluble at acidic pHs. Gel strengths can be evaluated using a texture analyzer such as the the TA.XT2, available from Texture Technologies.

A liquid ingestible composition can exhibit a viscosity in the range of from about 15 to about 200 cPs, or any value therebetween, at a shear rate of about 10^{-S}, e.g., about to about 24; about 20 to about 25, about 50 to 100, about 25 to 75, about 20 to 80, about 15 to about 20, about 100 to about 200, about 125 to about 175, about 150 to about 175 cPs. Viscosity can be measured by one having ordinary skill in the art, e.g., by measuring flow curves of solutions with increasing shear rate using a double gap concentric cylinder fixture (e.g., with a Parr Physica Rheometer).

A liquid ingestible composition can include a cation sequestrant, e.g., to prevent premature gelation of the anionic soluble fibers. A cation sequestrant can be selected from EDTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A cation sequestrant can be from about 0.001% to about 0.3% of the ingestible composition. Thus, for example, EDTA can be used at about 0.0015 to about 0.002% by weight and sodium citrate at about 0.230% to about 0.260% (e.g., 0.250%) by weight.

A liquid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passion fruit, blackberry, peach, mango, guava, pineapple, grapefruit, and others known to those having ordinary skill in the art. Vegetable juices for use include tomato, spinach, wheatgrass, cucumber, carrot, peppers, beet, aloe and others known to those of ordinary skill in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A liquid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and can include flavors such as kiwi, passion fruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources. Colorants can also be included depending on the final color to be achieved, in amounts quantum satis that can be determined by one having ordinary skill in the art.

Also provided are articles of manufacture that include a liquid ingestible composition. For example, a liquid ingestible composition can be provided in a container. Supplementary items such as straws, packaging, labels, etc. can also be included. In some cases, a source of one or more cations, as described below, can be included in an article of manufacture. For example, an article of manufacture can include a liquid ingestible composition in one container, and a source of cations in another container. Two or more containers may be attached to one another.

Sources of Cations

An ingestible composition can include a source of one or more cations. A source of one or more cations may be incorporated into an ingestible composition provided herein, or can consumed as a separate food article either before, after, or simultaneously with an ingestible composition.

A cation can be a monovalent or multivalent cation. In certain cases, a cation can be a monovalent cation, e.g., lithium, potassium, sodium, or rubidium. In some cases, a cation can be a multivalent cation, e.g., strontium, barium, calcium, magnesium, aluminum, manganese, iron, nickel, copper, zinc, bismuth, chromium, vanadium, and lanthanum. In certain compositions, one or more particular cations may be used with certain anionic soluble fibers, depending on the composition and gel strength desired. For example, for ingestible alginate compositions, calcium may be used to promote gelation. For gellan compositions, one or more of calcium, sodium, potassium, and magnesium may be used.

A source of one or more cations can be unable to, or be limited in its ability to, react with the one or more anionic soluble fibers in the ingestible composition until during or after ingestion. For example, physical separation of the source of the one or more cations from the one or more anionic soluble fibers, e.g., as a separate food article or in a separate matrix of the ingestible composition from the one or more anionic soluble fibers, can be used to limit the source of one or more cation's ability to react in other cases, the source of one or more cations is limited in its ability to react with the one or more anionic soluble fibers by protecting the source of one or more cations (see below) until during or after ingestion. Thus, a source of one or more cations, such as a protected source, can be included in the ingestible composition or can be included as a separate food article composition, e.g., for separate ingestion either before, during, or after ingestion of an ingestible composition. Typically, a separate food article containing the source of one or more cations would be consumed in a 4 hour time window flanking the ingestion of an ingestible composition containing the anionic soluble fiber. In certain cases, the window may be 3 hours, or 2 hours, or 1 hour. In other cases, the separate food article may be consumed immediately before or immediately after ingestion of an ingestible composition, e.g., within about 15 mins., such as within about 10 mins., 5 mins., or 2 mins. In other cases, a separate food article containing a source of one or more cations can be ingested simultaneously with an ingestible composition containing the one or more soluble anionic fibers, e.g., a snack chip composition where some chips include the source of one or more cations and some chips include the one or more soluble anionic fibers.

In one embodiment, a source of one or more cations can be included in an ingestible composition in a different food matrix from a matrix containing an anionic soluble fiber. For example, a source of one or more cations, such as a calcium salt, can be included in a separate matrix of a solid ingestible composition from the matrix containing the one or more soluble anionic fibers. Thus, physical separation of an anionic soluble fiber (e.g., within a snack bar or an extruded food product) from a source of one or more cations is also contemplated, such as by including the source of one or more cations in a matrix such as a frosting, coating, drizzle, chip, chunk, swirl, or interior layer. In one embodiment, a source of one or more cations, such as a protected source, can be included in a snack bar matrix that also contains an extruded crispy matrix that contains the anionic soluble fiber. In such a case, the source of one or more cations is in a separate matrix than the extruded crispy matrix containing the anionic soluble fiber. In another embodiment, a source of one or more cations can be included in a gel layer, e.g., a jelly or jam layer.

One source of cations are cation salts. Typically, a cation salt can be selected from the following salts: citrate, tartrate, malate, formate, lactate, gluconate, phosphate, carbonate, sulfate, chloride, acetate, proprionate, butyrate, caprylate, valerate, fumarate, adipate, and succinate.

In certain cases, a cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, dicalcium phosphate dihydrate, anhydrous calcium diphosphate, dicalcium phosphate anhydrous, tricalcium phosphate, calcium carbonate, calcium sulfate dihydrate, calcium sulfate anhydrous, calcium chloride, calcium acetate monohydrate, monocalcium phosphate monohydrate, and monocalcium phosphate anhydrous.

A source of one or more cations can be a protected source of one or more cations. As used herein, the term “protected” means that the source has been treated in such a way to delay (e.g., until during or after ingestion or until a certain pH range has been reached) reaction of the one or more cations with the anionic soluble fiber as compared to a salt form of the one or more cations.

A number of methods can be used to protect a source of one or more cations. For example, an enteric coating can prevent the solubilization of the source of one or more cations in the stomach so that solubilization can then occur at the neutral pH range of the intestine. An enteric coating can provide for prolonged release or delayed pulse solublization of the source of one or more cations, or can may be designed to provide for a combination of immediate, pulsed and/or prolonged delivery. The enteric coating composition can be a member selected from the group consisting of cellulose acetyl phthalate, cellulose diacetyl phthalate, cellulose triacetyl phthalate, cellulose acetate phthalate, hydroxypropylmethylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl comprises from 1 to 7 straight and branched alkyl groups, aryl phthalates, and the like. Other entering coating polymers include Eudragit materials (Rohm Pharma), and Acryl EZE MP (Colorcon, Inc, West Point, Pa.). Agents that slow the dissolution of the source after dissolution of the enteric coating can also be included. Non-limiting examples of these materials are methylcellulose, ethylcellulose, propylcellulose, and starch or starch derivatives. Enteric polymers other than the materials traditionally used for enteric coatings in the pharmaceutical industry can also be used, such as modified or substituted starches, chitosan, and hydrophobic proteins such as maize zein.

Enteric particles of a wide range of diameters and shapes can be formed that contain suitable sources of cations.

In some cases, microparticles or nanoparticles comprising double or multiple emulsions, such as water/oil/water (“w/o/w”) or oil/water/oil (“o/w/o”) emulsions, of a cation and an anionic soluble fiber can be used to protect a source of one or more cations. In one embodiment, a calcium alginate microparticle or nanoparticle is used. For example, a calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 uM (e.g., about 5 to about 10 uM, or about 3 to about 8 uM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art, see the Examples, below.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a liquid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of one or more cations can be included in an ingestible composition. For example, a calcium-containing liposome can be used. The preparation of liposomes containing cations is well known to those having ordinary skill in the art; see ACS Symposium Series, 1998 709:203-211; Chem. Mater. 1998 (109-116). Cochleates can also be used, e.g., as described in U.S. Pat. No. 6,592,894 and U.S. Pat. No. 6,153, 217. The creation of cochleates using cations such as calcium can protect the cations from reacting with the anionic soluble fiber within the aqueous phase of an ingestible composition, e.g., by wrapping the cations in a hydrophobic lipid layer, thus delaying reaction with the fiber until digestion of the protective lipids in the stomach and/or small intestine via the action of lipases.

In certain cases, a cation-containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligo-saccharides using methods known to those having ordinary skill in the art; see, e.g., WO 02/05667. A carbohydrate glass can be used, e.g., in a coating or within a food matrix.

Fiber Blends

A fiber blend provided herein can include one or more anionic soluble fibers. For example, a fiber blend can include alginate and pectin. An alginate for use in the blend can include both intermediate and low molecular weight distribution range forms. A ratio of total alginate to total pectin in a blend can be from about 8:1 to about 5:1, or any value therebetween, such as about 7:1, about 6.5:1, about 6.2:1, or about 6.15:1. A ratio of an intermediate molecular weight alginate to a low molecular weight alginate can range from about 0.65:1 to about 2:1, or any value therebetween (e.g., about 0.75:1; about 1:1; about 1.25:1; about 1.5:1; or about 1.75:1). In certain cases, a ratio of an intermediate molecular weight alginate to a low molecular weight alginate is about 0.8:1 to about 0.9:1.

Methods of Facilitating Satiety, Increasing Viscosity of Stomach and/or Small Intestine Contents, and Promoting Weight Loss

Provided herein are methods employing the ingestible compositions described herein. The ingestible compositions can be ingested in connection with any of the following methods: treatment or prevention of obesity; assistance in dietary compliance; reduction of body weight; reduction of caloric intake (daily or per meal); suppression of appetite; glycemic control; weight maintenance; and/or increasing the viscosity of the stomach and/or small intestine contents;.

For example, a method of facilitating satiety and/or satiation in an animal is provided. The method can include providing an ingestible composition to an animal. An animal can be any animal, including a human, monkey, mouse, rat, snake, cat, dog, pig, cow, sheep, horse, bird, or horse. The step of providing can include providing the ingestible combination either alone or in combination with other meal items. Providing can include co-providing, either before, after, or during provision of the ingestible composition, a source of one or more cations, such as calcium or a sequestered source of calcium, as described herein. The source of one or more cations can be provided within about a four hour time window flanking the provision of the ingestible composition. For example, a source of calcium, such as a solution of calcium lactate, can be provided to an animal immediately after the animal has ingested a liquid ingestible composition as provided herein. Satiety and/or satiation can be evaluated using consumer surveys (e.g., for humans) that can demonstrate a statistically significant measure of increased satiation and/or satiety. For example, a VARS (Visual Analogue Rating Scale) questionnaire can be used to determine a number of satiety parameters (fullness, hunger, appetite), as described in U.S. Pat. Publication 2005/0170059. Alternatively, data from paired animal sets showing a statistically significant reduction in total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include providing an ingestible composition to an animal. An animal can be any animal, as described above, and provision can be as described previously. Viscosity of stomach contents can be measured by any method known to those having ordinary skill in the art, including endoscopic techniques, imaging techniques (e.g., MRI, PET, density scans), or in vivo or ex vivo viscosity measurements in e.g., control and treated animals.

Also provided are methods for promoting weight loss by providing an ingestible composition as provided herein to an animal. Provision can be as described previously. The amount and duration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

### EXAMPLES

1. Gel Strength—Static Gel Strength Assays

A static gel screening technique was developed to assess the gel strength of anionic soluble fibers and fiber blends (in the presence and absence of cations) and without shear and enzymes. The method lowers the pH slowly, and adds a source of cations (e.g., calcium) gradually to make uniform gels. For example, glucono delta lactone (GDL) can be used as an acidulant, as it dissociates to lower the pH gradually. Dicalcium phosphate is a slowly soluble calcium source, which becomes more soluble at acidic pHs. Gel strengths are then evaluated using a texture analyzer, the TA.XT2, available from Texture Technologies.

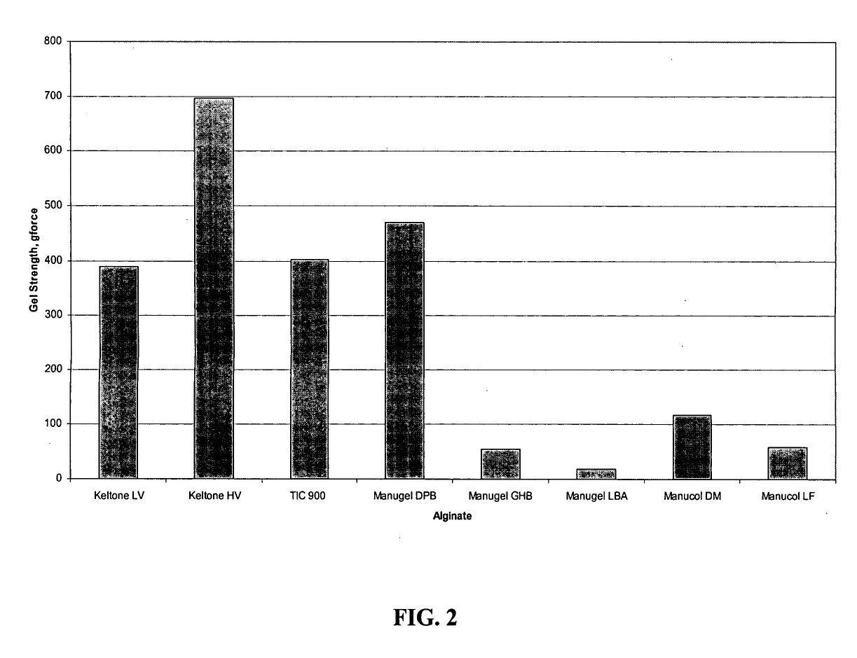

One percent fiber solutions were prepared and gel strength evaluated as follows:
1. 1.3 grams of dicalcium phosphate anhydrous (DCPA) was blended with 10 g of sugar (sucrose).
2. 304.5 grams of water were placed in a blender, and 0.2 g of sodium hexametaphosphate added while stirring for about 30 seconds.
3. 4 grams of the fiber or mixture of fibers was added to the blender and blended for an additional 120 seconds, then the mixture was poured into a beaker.
4. The mixture was heated just to boiling with a stir bar on a hot place; heat was stopped but stirring was continued.
5. The DCPA and sugar solution was added to the stirring mixture for an additional 30 seconds.
6. 20 g of glucono delta lactone was mixed 60 g of water until dissolved and added to the hot mixture.
7. The resulting mixture was aliquoted into 25 ml portions in hexagonal weigh boats while hot.
8. The aliquots were allowed to cool overnight.
9. Gel strength was evaluated using the texture analyzer. See FIG. 2.
   2. Crispy Formulations

A variety of crispy formulations were prepared using the formulations as shown below followed by extrusion (under heat and high-pressure) to make crispies:

Batch 1A-5367-54-01A

| | Ingredients | % |
|---|---|---|
| 1 | Rice Flour | 52.30 |
| 2 | Alginate LBA | 25.20 |
| 3 | Whey Protein Isolate | 20.00 |
| 4 | Starch | 2.00 |
| 5 | Salt | 0.50 |
| | Total | 100.00 |

Batch 2, 5367-54-02

| | Ingredients | % |
|---|---|---|
| 1 | Rice Flour | 54.30 |
| 2 | Alginate LBA | 25.20 |
| 3 | Whey Protein Isolate | 12.00 |
| 4 | Starch | 8.00 |
| 5 | Salt | 0.50 |
| | Total | 100.00 |

Batch 3, 5367-54-03

| | Ingredients | % |
|---|---|---|
| 1 | Rice Flour | 66.30 |
| 2 | Alginate LBA | 25.20 |
| 3 | Whey Protein Isolate | 00.00 |
| 4 | Starch | 8.00 |
| 5 | Salt | 0.50 |
| | Total | 100.00 |

Batch 4, 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate | 12.00 |
| 6 | Starch | 8.00 |
| 7 | Salt | 0.50 |
| | Total | 100.00 |

Batch #1, repeat 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate BiPro | 12.00 |
| 6 | Wheat Starch | 8.00 |
| 7 | Salt | 0.50 |
| | Total | 100.00 |

Batch #2, based on 5367-54-04
Formula # 5981-04-01

| | Ingredients | % |
|---|---|---|
| 1 | Rice Flour | 54.30 |
| 2 | Alginate DPB | 25.20 |
| 3 | Whey Protein Isolate BiPro | 12.00 |
| 4 | Whey Starch | 8.00 |
| 5 | Salt | 0.50 |
| | Total | 100.00 |

Batch #3, Same as Batch #2 (5981-04-01),
replacing DPB with KTHV
Formula # 5981-04-02

| | Ingredients | % |
|---|---|---|
| 1 | Rice Flour | 54.30 |
| 2 | Alginate KTHV | 25.20 |
| 3 | Whey Protein Isolate BiPro | 12.00 |
| 4 | Whey Starch | 8.00 |
| 5 | Salt | 0.50 |
| | Total | 100.00 |

Batch #4: Based on 5981-04-01, increased alginate by 25%
Formula # 5981-04-03

| | Ingredients | % |
|---|---|---|
| 1 | Rice Flour | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Batch #5: Based on 5981-04-01, increased alginate by 50%
Formula # 5981-04-04

| | Ingredients | % |
|---|---|---|
| 1 | Rice Flour | 49.70 |
| 2 | Alginate KTHV | 37.80 |
| 3 | Whey Protein Isolate BiPro | 8.00 |
| 4 | Corn Starch | 4.00 |
| 5 | Salt | 0.50 |
| | Total | 100.00 |

Formula # 5981-04-05

| | Ingredients | % |
|---|---|---|
| 1 | Rice Flour | 46.00 |
| 2 | Alginate KTHV | 31.50 |
| 3 | Whey Protein Isolate BiPro | 8.00 |
| 4 | Corn Starch | 4.00 |
| 5 | Salt | 0.50 |
| 6 | Inulin | 10.00 |
| | Total | 100.00 |

Formula # 5981-04-06

| | Ingredients | % |
|---|---|---|
| 1 | Corn Meal | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Formula # 5981-04-07

| | Ingredients | % |
|---|---|---|
| 1 | Corn Cone | 64.00 |
| 2 | Alginate KTHV | 31.50 |
| 3 | Whey Protein Isolate BiPro | 8.00 |
| 4 | Corn Starch | 0.00 |
| 5 | Salt | 0.50 |
| 6 | Inulin F97 | 0.00 |
| | Total | 100.00 |

Formula # 5981-04-08

| | Ingredients | % |
|---|---|---|
| 1 | Corn Cone | 53.70 |
| 2 | Alginate KTHV | 37.80 |
| 3 | Whey Protein Isolate BiPro | 0.00 |
| 4 | Corn Starch | 0.00 |
| 5 | Salt | 0.50 |
| 6 | Inulin F97 | 8.00 |
| | Total | 100.00 |

Batch #10
Formula # 5981-04-09

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch #11
Formula # 5981-04-10

| | Ingredients | % |
|---|---|---|
| 1 | Corn Cone | 49.50 |
| 2 | Alginate KTHV | 50.00 |
| 3 | Whey Protein Isolate BiPro | 0.00 |
| 4 | Corn Starch | 0.00 |
| 5 | Salt | 0.50 |
| 6 | Inulin F97 | 0.00 |
| | Total | 100.00 |

Batch #12, same formula as 5981-04-07, used different die to make curls
Formula # 5981-04-11

| | Ingredients | % |
|---|---|---|
| 1 | Corn Cone | 56.00 |
| 2 | Alginate KTHV | 31.50 |
| 3 | Whey Protein Isolate BiPro | 4.00 |
| 4 | Corn Starch | 0.00 |
| 5 | Salt | 0.50 |
| 6 | Inulin F97 | 8.00 |
| | Total | 100.00 |

Batch #13 = batch #10 5981-04-09
Formula # 5981-04-12

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch # 14: same as batch #4, 5981-04-03
except replaced KTHV with DPB
Formula # 5981-15-01

| | Ingredients | % |
|---|---|---|
| 1 | Rice Flour | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #15: based on batch #14
Formula # 5981-15-02

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Cocoa | 5.00 |
| | Total | 100.00 |

Batch #16: based on batch #14
Formula # 5981-15-03

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 4.00 |
| 5 | Salt | 0.50 |
| 6 | Micro-crystalline Cellolose | 4.00 |
| | Total | 100.00 |

Batch #17: based on batch 14
Formula # 5981-15-04

| | Ingredients | % |
|---|---|---|
| 1 | Rice Flour | 56.00 |
| 2 | Alginate DPB | 31.50 |
| 3 | Whey Protein Isolate BiPro | 7.00 |
| 4 | Corn Starch | 4.00 |
| 5 | Salt | 0.50 |
| 6 | Glycerine | 1.00 |
| | Total | 100.00 |

Batch #18: based on batch # 14
Formula # 5981-15-05

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Oil | 5.00 |
| | Total | 100.00 |

Batch #19: based on batch # 14
Formula # 5981-15-06

| | Ingredients | % |
|---|---|---|
| 1 | Rice Flour | 56.00 |
| 2 | Alginate DPB | 31.50 |
| 3 | Whey Protein Isolate BiPro | 7.95 |
| 4 | Corn Starch | 4.00 |
| 5 | Salt | 0.50 |
| 6 | Sucrolose | 0.05 |
| | Total | 100.00 |

Batch #20: based on batch # 14
Formula # 5981-15-07

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Trehalose | 5.00 |
| | Total | 100.00 |

Batch #21: based on batch # 7, 5981-04-06
Formula # 5981-15-08

| | Ingredients | % |
|---|---|---|
| 1 | Corn Cone | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #22: same as batch # 21
Formula # 5981-15-09

| | Ingredients | % |
|---|---|---|
| 1 | Corn Cone 2 | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #25
Formula # 5981-15-12

| | Ingredients | % |
|---|---|---|
| 1 | Corn Cone | 54.00 |
| 2 | Alginate DPB | 31.50 |
| 3 | Whey Protein Isolate BiPro | 2.00 |
| 4 | Corn Starch | 2.00 |
| 5 | Salt | 0.50 |
| 6 | Corn Fiber | 10.00 |
| | Total | 100.00 |

Formula # 5981-15-15

| | Ingredients | % |
|---|---|---|
| 1 | Rice Flour | 56.50 |
| 2 | Alginate DPB | 31.50 |
| 3 | Whey Protein Isolate BiPro | 4.00 |
| 4 | Corn Starch | 3.00 |
| 5 | Fractionated Canola Oil | 5.00 |
| | Total | 100.00 |

Formula # 5981-15-25

| | Ingredients | % |
|---|---|---|
| 1 | Rice Flour | 88.00 |
| 2 | Whey Protein Isolate BiPro | 4.00 |
| 3 | Corn Starch | 3.00 |
| 4 | Fractionated Canola Oil | 5.00 |
| | Total | 100.00 |

3. Bar Formulations

A variety of bar formulations incorporating various crispy formulations set forth above were prepared as shown below. Typically, the ingredients can be mixed (e.g., in the order set forth), and then formed, cold-extruded, or cut into the desired shapes. In certain embodiments, nutritional bars with a nougat center can be prepared by mixing all the liquid ingredients in a mixer bowl, e.g., with a paddle attachment for about 1 minute; adding all dry ingredients except proteins and mixing (e.g., on low speed) for an additional minute; adding proteins to mixing (e.g., on medium speed for an additional 2 minutes). The dough can then be formed into desired shapes and sizes either manually or through an extruder (e.g., cold extrusion). Solid ingestible compositions such as bars or cookies can be coated or frosted with coatings or frostings of desired flavors and/or colors by submersion into melted (e.g., 120° F.) compound coating, or in to chocolate that has been melted (e.g., 120° F.) and tempered (90° F.). Coated compositions can be allowed to cool and may then be packaged.

Formula 5367-52-01

| # | Ingredients | % in Bar |
|---|---|---|
| 1 | High Maltose Corn Syrup | 15.36 |
| 2 | HFCS | 4.80 |
| 3 | Molasses | 0.64 |
| 4 | Honey | 0.64 |
| 5 | Granulated Sugar | 4.16 |
| 6 | Salt | 0.32 |
| 7 | Citric Acid | 0.06 |

Step 1: Mix well, cook to brix at 88.5%

| | | |
|---|---|---|
| 8 | Erythritol | 2.18 |
| 9 | Inulin | 0.00 |
| 10 | Calcium Carbonate | 1.60 |

Step 2: Add dry ingredients slowly to syrup above, mix well using high
shear mixer

| | | |
|---|---|---|
| 11 | Canola Oil | 1.28 |
| 12 | Lecithin | 0.32 |
| 13 | Vanilla Flavor | 0.48 |
| 14 | Cranberry Flavor | 0.16 |

Step 3, Add rest of liquid ingredients, mix well. Using high shear mixer

| | | |
|---|---|---|
| | Total | 32.00 |
| 15 | Alginate Crisps (5367-54-00) | 38.00 |
| 16 | Oats | 10.0 |
| 17 | Whole Almond | 8.0 |
| 18 | Raisins | 6.0 |
| 19 | Sweetened Cranberry | 6.0 |

Step 4: Add binder to dry ingredients, mix well quickly
Step 5: Add the mass to a pan, roll to right density, cool at refrigerator for
20 min, cut to desired size

| | |
|---|---|
| Total | 100.00 |

Formula 5367-52-02
Based on 5367-52-01, replaced calcium carbonate with inulin

| # | Ingredients | % in Bar |
|---|---|---|
| 1 | High Maltose Corn Syrup | 15.36 |
| 2 | HFCS | 4.80 |
| 3 | Molasses | 0.64 |
| 4 | Honey | 0.64 |
| 5 | Granulated Sugar | 4.16 |
| 6 | Salt | 0.32 |
| 7 | Citric Acid | 0.06 |

Step 1: Mix well, cook to brix at 88.5%

| | | |
|---|---|---|
| 8 | Erythritol | 2.18 |
| 9 | Inulin | 1.60 |
| 10 | Calcium Carbonate | 0.00 |

Step 2: Add dry ingredients slowly to syrup above, mix well using high
shear mixer

| | | |
|---|---|---|
| 11 | Canola Oil | 1.28 |
| 12 | Lecithin | 0.32 |
| 13 | Vanilla Flavor | 0.48 |
| 14 | Cranberry Flavor | 0.16 |

Step 3, Add rest of liquid ingredients, mix well. Using high shear
mixer

| | | |
|---|---|---|
| | Total | 32.00 |
| 15 | Alginate Crisps (5367-54-00) | 38.00 |
| 16 | Oats | 10.0 |
| 17 | Whole Almond | 8.0 |
| 18 | Raisins | 6.0 |
| 19 | Sweetened Cranberry | 6.0 |

Step 4: Add binder to dry ingredients, mix well quickly
Step 5: Add the mass to a pan, roll to right density, cool at refrigerator for
20 min, cut to desired size

| | |
|---|---|
| Total | 100.00 |

Formula 5981-07-01

| # | Ingredients | % in Bar |
|---|---|---|
| 1 | High Maltose Corn Syrup | 15.36 |
| 2 | HFCS | 4.80 |
| 3 | Granulated Sugar | 3.84 |
| 4 | Erythritol | 3.14 |
| 5 | Fructose | 1.92 |
| 6 | Canola Oil | 0.64 |
| 7 | Molasses | 0.48 |
| 8 | Honey | 0.48 |
| 9 | Salt | 0.48 |
| 10 | Vanilla Flavor | 0.48 |
| 11 | Lecithin | 0.16 |
| 12 | Cranberry Flavor | 0.16 |
| 13 | Citric Acid | 0.06 |
| 14 | Calcium Carbonate | 0.00 |
| | Total | 32.00 |

Step 1: Mix all ingredients except flavors, oil and lecithin, cook
at 180 to 200 Brix 88%
Step 2: Add flavors and oil and lecithin, check Brix to 87%

| | | |
|---|---|---|
| 15 | Alginate Crisps (Batch #4, 5981-04-03) | 39.7 |
| 16 | Oats | 8.0 |
| 17 | Whole Almond | 7.3 |
| 18 | Raisins | 7.0 |
| 19 | Sweetened Cranberry | 6.0 |

Step 3, Add 640 g syrup to dry ingredients, mix well quickly
Step 4: Transfer mass to a pan, roll it flat, cool down in refrigerator for
minimum 15 min
Step 5: Cut to L 3.5″, W 1.2″ and H 0.8″, wrap them

| | |
|---|---|
| Total | 100.00 |

Results:
1) Good tasting bar, loosely bound, not sticky.

Formula 5981-07-02

| # | Ingredients | % in Bar |
|---|---|---|
| 1 | High Maltose Corn Syrup | 15.36 |
| 2 | HFCS | 4.80 |
| 3 | Granulated Sugar | 3.36 |
| 4 | Fructose | 1.92 |
| 5 | Canola Oil | 0.64 |
| 6 | Molasses | 0.48 |
| 7 | Honey | 0.48 |
| 8 | Salt | 0.48 |
| 9 | Vanilla Flavor | 0.48 |
| 10 | Lecithin | 0.16 |
| 11 | Cranberry Flavor | 0.16 |
| 12 | Citric Acid | 0.06 |
| 13 | Monocalcium Phosphate, Monohydrate | 3.62 |
| | Total | 32.00 |

Step 1: Mix all ingredients except flavors, oil and lecithin, cook at
180 to 200 Brix 88%
Step 2: Add flavors, oil and lecithin, mix well, check Brix to 87%

| | | |
|---|---|---|
| 14 | Alginate Crisps (Batch #4, 5981-04-03) | 45.4 |
| 15 | Oats | 0 |
| 16 | Whole Almond | 7.6 |
| 17 | Raisins | 7.0 |
| 18 | Sweetened Cranberry | 8.0 |

Step 3, Add 640 g syrup to dry ingredients, mix quickly
Step 4: Transfer the mass to a pan, roll it flat, cool down in refrigerator for
minimum 15 min
Step 5: Cut to L 3.5″, W 1.2″ and H 0.8″, then wrap

| | |
|---|---|
| Total | 100.00 |

Formula 5981-07-03

| # | Ingredients | % in Bar |
|---|---|---|
| 1 | High Maltose Corn Syrup | 15.36 |
| 2 | HFCS | 5.34 |
| 3 | Granulated Sugar | 3.36 |
| 4 | Fructose | 1.92 |
| 5 | Canola Oil | 0.64 |
| 6 | Molasses | 0.48 |
| 7 | Honey | 0.48 |
| 8 | Salt | 0.48 |
| 9 | Vanilla Flavor | 0.48 |
| 10 | Lecithin | 0.16 |
| 11 | Cranberry Flavor | 0.16 |
| 12 | Citric Acid | 0.06 |
| 13 | Dicalcium Phosphate Dihydrate | 3.07 |
| | Total | 32.00 |

Step 1: Mix all ingredients except flavors, oil and lecithin, cook
at 180 to 200 Brix 88%
Step 2: Add flavors, oil and lecithin, mix well, check Brix to 87%

| | | |
|---|---|---|
| 15 | Alginate Crisps (Batch #4, 5981-04-03) | 45.4 |
| 16 | Oats | 0 |
| 17 | Whole Almond | 7.6 |
| 18 | Raisins | 7.0 |
| 19 | Sweetened Cranberry | 8.0 |

Step 3, Add 640 g syrup to dry ingredients, mix quickly
Step 4: Transfer the mass to a pan, roll it flat, cool down in refrigerator
for minimum 15 min
Step 5: Cut to L 3.5″, W 1.2″ and H 0.8″, then wrap

| | |
|---|---|
| Total | 100.00 |

Formula 5981-07-04

| # | Ingredients | % in Bar |
|---|---|---|
| 1 | High Maltose Corn Syrup | 16.32 |
| 2 | HFCS | 5.81 |
| 3 | Granulated Sugar | 2.38 |
| 4 | Fructose | 2.04 |
| 5 | Molasses | 0.68 |
| 6 | Honey | 0.51 |
| 7 | Salt | 0.51 |
| 8 | Dicalcium Phosphate Dihydrate | 5.00 |

Step 1: Weigh and cook above ingredients at 180 to 88% Brix

| | | |
|---|---|---|
| 9 | Vanilla Flavor | 0.51 |
| 10 | Cranberry Flavor | 0.17 |
| 11 | Citric Acid | 0.07 |
| | Total | 34.00 |

Step 2: Add flavors, mix well, and cook gently, check Brix to 87%

| | | |
|---|---|---|
| 12 | Alginate Crisps (Batch #4, 5981-04-03) | 32.0 |
| 13 | Rolled Oats | 14.0 |
| 14 | Whole Almond | 6.0 |
| 15 | Raisins | 7.0 |
| 16 | Sweetened Cranberry | 8.0 |

Step 3, Add 680 g syrup to dry ingredients, mix quickly
Step 4: Transfer the mass to a pan, roll it flat, cool down in refrigerator for
minimum 15 min
Step 5: Cut to L 3.5″, W 1.2″ and H 0.8″, then wrap

| | |
|---|---|
| Total | 100.00 |

Formula 5981-07-06

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 03.06 |
| 6 | Salt | 0.51 |
| 7 | Dicalcium Phosphate Dihydrate | 5.00 |

Step 2: Add all dry ingredients, cook Brix to 88% (using the new one)

| | | |
|---|---|---|
| 8 | Canola Oil | 0.68 |
| 9 | Vanilla Flavor | 0.51 |
| 11 | Cranberry Flavor | 0.17 |
| 12 | Citric Acid | 0.07 |
| | Total | 34.00 |

Step 3: Add flavors, citric acid and oil, mix well and cook gently, check
Brix to 87%

| | | |
|---|---|---|
| 15 | Alginate Crisps (Batch # 18, 5981-15-05) | 32 |
| 16 | Oats | 14 |
| 17 | Whole Almond | 6 |
| 18 | Raisins | 7 |
| 19 | Sweetened Cranberry | 7 |

Step 4: Add 68 g syrup to dry ingredients, mix quickly
Step 5: Transfer the mass to a pan, roll it flat, cool down in refrigerator for
minimum 15 min
Step 6: Cut to L 3.5″, W 1.2″ and H 0.8″, then wrap

| | |
|---|---|
| Total | 100 |

Formula 5981-07-06

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 3.06 |
| 6 | Salt | 0.51 |
| 7 | Dicalcium Phosphate Dihydrate | 5.00 |

Step 2: Add all dry ingredients, cook Brix to 88% (using the new one)

| | | |
|---|---|---|
| 8 | Canola Oil | 0.68 |
| 9 | Vanilla Flavor | 0.51 |
| 10 | Cranberry Flavor | 0.17 |
| 11 | Citric Acid | 0.07 |
| | Total | 34.00 |

Step 3: Add flavors, citric acid and oil, mix well and cook gently, check
Brix to 87%

| | | |
|---|---|---|
| 12 | Alginate Crisps (Batch # 18, 5981-15-05) | 32 |
| 13 | Oats | 14 |
| 14 | Whole Almond | 6 |
| 15 | Raisins | 7 |
| 16 | Sweetened Cranberry | 7 |

Step 4: Add 680 g syrup to dry ingredients, mix quickly
Step 5: Transfer the mass to a pan, roll it flat, cool down in refrigerator for
minimum 15 min
Step 6: Cut to L 3.5″, W 1.2″ and H 0.8″, then wrap

| | |
|---|---|
| Total | 100.00 |

Formula 5981-07-07, Serving size: 30 g

| # | Ingredients | % in Bar |
|---|---|---|
| 1 | High Maltose Corn Syrup | 15.64 |
| 2 | HFCS | 5.81 |
| 3 | Molasses | 1.02 |

Step 1: Weigh and cook all above liquid at 160° F.

| | | |
|---|---|---|
| 4 | Maltodextrin | 1.53 |
| 5 | Fructose | 3.06 |
| 6 | Salt | 0.51 |
| 7 | Dicalcium Phosphate Dihydrate | 5.00 |

Step 2: Add all dry ingredients, cook Brix to 87%

| | | |
|---|---|---|
| 8 | Canola Oil | 0.68 |
| 9 | Vanilla Flavor | 0.51 |
| 10 | Cranberry Flavor | 0.17 |
| 11 | Citric Acid | 0.07 |
| | Total | 34.00 |

Step 3: Add flavors, citric acid and oil, mix well and cook gently, check
Brix to 85%

| | | |
|---|---|---|
| 12 | Alginate Crisps (Batch # 18, 5981-15-05) | 22 |
| 13 | Alginate Crisps (Crushed) | 10 |
| 14 | Rolled Oats | 13 |
| 15 | Whole Almond | 5 |
| 16 | Raisins | 8 |
| 17 | Sweetened Cranberry | 8 |

Step 4: Add 680 g syrup to dry ingredients, mix quickly
Step 5: Transfer the mass to a pan, roll it flat, cool down in refrigerator for
minimum 15 min
Step 6: Cut to L 3.5″, W 1.375″ and H 0.8″, then wrap

| | |
|---|---|
| Total | 100.00 |

Formula 5981-07-08, Serving size: 30 g

| # | Ingredients | % in Bar |
|---|---|---|
| 1 | High Maltose Corn Syrup | 14.45 |
| 2 | HFCS | 5.81 |
| 3 | Molasses | 1.02 |

Step 1: Weigh and cook all above liquid at 160° F.

| | | |
|---|---|---|
| 4 | Fructose | 3.06 |
| 5 | Calcium Lacatate | 8.98 |

Step 2: Add all dry ingredients, cook Brix to 86%

| | | |
|---|---|---|
| 9 | Vanilla Flavor | 0.51 |
| 10 | Cranberry Flavor | 0.17 |
| | Total | 34.00 |

Step 3: Add flavors, citric acid and oil, mix well and cook gently, check
Brix to 84.5%

| | | |
|---|---|---|
| 12 | Alginate Crisps (Batch # 18, 5981-15-05) | 16 |
| 13 | Alginate Crisps (Crushed) | 16 |
| 14 | Rolled Oats | 13 |
| 15 | Whole Almond | 5 |
| 16 | Raisins | 8 |
| 17 | Sweetened Cranberry | 8 |

Step 4: Add 340 g syrup to dry ingredients, mix quickly
Step 5: Transfer the mass to a pan, roll it flat, cool down in refrigerator for
minimum 15 min
Step 6: Cut to L 3.5″, W 1.375″ and H 0.8″, then wrap

| | |
|---|---|
| Total | 100.00 |

Modified 5981-07-07

| # | Ingredients | % in Bar |
|---|---|---|
| 1 | High Maltose Corn Syrup | 19.83 |
| 2 | HFCS | 2.71 |
| 3 | Molasses | 1.07 |

Step 1: Weigh and cook all above liquid at 160° F.

| | | |
|---|---|---|
| 4 | Maltodextrin | 0.00 |
| 5 | Fructose | 3.75 |
| 6 | Sugar | 1.61 |
| 7 | Dicalcium Phosphate Dihydrate | 3.57 |
| 8 | Citric Acid | 0.07 |

Step 2: Add all dry ingredients, cook Brix to 88%

| | | |
|---|---|---|
| 9 | Canola Oil | 0.71 |
| 10 | Vanilla Flavor | 0.54 |
| 11 | Cranberry Flavor | 0.14 |
| | Total | 34.00 |

Step 3: Add flavors and oil, mix well and cook gently, check Brix to 87%

| | | |
|---|---|---|
| 12 | Test Crisps (5981-15-15) | 33.10 |
| 13 | Rolled Oats | 18 |
| 14 | Raisins | 4 |
| 15 | Cranberry Halves | 10.90 |

Step 4: Add 680 g syrup to dry ingredients, mix quickly
Step 5: Transfer the mass to a pan, roll it flat, cool down in refrigerator for
minimum 15 min
Step 6: Cut to L 4.0″, W 1.55″ and H 0.8″, then wrap

| | |
|---|---|
| Total | 100.00 |

Additional bar formulations that did not include crispies were prepared as described below. Typically, the ingredients can be mixed (e.g., in the order set forth), and then formed, cold-extruded, or cut into the desired shapes. In certain embodiments, nutritional bars with a nougat center can be prepared by mixing all the liquid ingredients in a mixer bowl, e.g., with a paddle attachment for about 1 minute; adding all dry ingredients except proteins and mixing (e.g., on low speed) for an additional minute; adding proteins to mixing (e.g., on medium speed for an additional 2 minutes). The dough can then be formed into desired shapes and sizes either manually or through an extruder (e.g., cold extrusion). Solid ingestible compositions such as bars or cookies can be coated or frosted with coatings or frostings of desired flavors and/or colors by submersion into melted (e.g., 120° F.) compound coating, or in to chocolate that has been melted (e.g., 120° F.) and tempered (90° F.). Coated compositions can be allowed to cool and may then be packaged.

Chocolate Peanut Butter
Serving size: 50 g

| # | Ingredient | Percentage |
|---|---|---|
| 1 | Chocolate Coating | 12.50 |
| 2 | HFCS | 20.00 |
| 3 | Glycerine | 12.50 |
| 4 | Water | 10.00 |
| 5 | Canola Oil | 5.00 |
| 6 | Inulin | 5.00 |
| 7 | Tricalcium Phosphate | 2.50 |
| 8 | Calcium Caseinate | 10.00 |
| 9 | Whey Protein Isolate | 12.50 |
| 10 | Psyllium | 10.00 |
| | Total | 100.00 |

I. Chocolate Peanut Butter
Formula # 5367-45-33
Serving size 55 g, 5 g alginate

| # | Ingredient | Percentage |
|---|---|---|
| 1 | Sugar-Free Choc Coating | 18.20 |
| 2 | HFCS | 13.00 |
| 3 | Maltitol | 9.90 |
| 4 | Glycerine | 5.00 |
| 5 | Peanut Butter | 3.00 |
| 6 | Canola Oil | 3.00 |
| 7 | Peanut Butter Flavor | 3.00 |
| 8 | Vanilla | 0.50 |
| | Mix for 0.5 min | |
| 9 | Alginate | 9.10 |
| | Mix for 1 min | |
| 10 | Erythritol | 8.00 |
| 11 | Inulin | 3.00 |
| 12 | Peanut Flour | 5.00 |
| | Mix for 0.5 min | |
| 13 | Tricalcium Phosphate | 0.00 |
| 14 | Calcium Carbonate | 0.00 |
| 15 | Whey Protein Isolate | 8.30 |
| 16 | Peanuts | 11.00 |
| | Mix for 1 min | |
| | Total | 100.00 |

II. Chocolate Peanut Butter
A. Formula # 5367-45-32
Serving size 55 g, 5 g alginate

| # | Ingredient | Percentage |
|---|---|---|
| 1 | Sugar-Free Choc Coating | 18.20 |
| 2 | HFCS | 12.00 |
| 3 | Maltitol | 9.90 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.00 |
| | Mix for 0.5 min | |
| 14 | Tricalcium Phosphate | 0.00 |
| 15 | Calcium Carbonate | 0.00 |
| 16 | Whey Protein Isolate | 8.30 |
| 17 | Peanuts | 11.00 |
| | Mix for 1 min | |
| | Total | 100.00 |

Aw = 0.521

Chocolate Peanut Butter
Formula # 5367-45-01
Serving size 45 g, contains 200 mg Ca, 5% alginate/pectin, 3 g inulin

| # | Ingredient | Percentage |
|---|---|---|
| 1 | Chocolate Coating | 15.00 |
| 2 | HFCS | 9.50 |
| 3 | Water | 9.50 |
| 4 | Glycerine | 7.00 |
| 5 | Peanut Flour | 8.00 |
| 6 | Peanut Butter | 10.00 |
| 7 | Peanut Flavor | 1.15 |
| 8 | Inulin | 6.70 |
| 9 | Alginate | 2.50 |
| 10 | Pectin | 2.50 |
| 11 | Soy Protein Isolate | 8.00 |
| 12 | Calcium Caseinate | 7.00 |
| 13 | Whey Protein Isolate | 7.00 |
| 14 | Peanuts | 6.15 |
| 15 | Tricalcium Phosphate | 1.15 |
| | Total | 100.00 |

III. Chocolate Raspberry
Formula # 5367-44-03
Serving size 40 g, contains 200 mg Ca, 1 g alginate, 1 g pectin, 3 g inulin

| # | Ingredient | Percentage |
|---|---|---|
| 1 | Sugar-Free Choc Coating | 18.00 |
| 2 | Water | 12.60 |
| 3 | Glycerine | 8.00 |
| 4 | Canola Oil | 7.00 |
| 5 | Raspberry Flavor | 1.50 |
| 6 | Raspberry Flavor | 0.75 |
| 7 | White Chocolate Flavor | 0.35 |
| 8 | Vanilla Flavor | 1.00 |
| 9 | Sucrolose | 0.20 |
| 10 | Red Color | 0.10 |
| 11 | Inulin | 5.45 |
| 12 | Erythritol | 8.00 |
| 13 | Fructose | 4.00 |
| 14 | Malic Acid | 0.30 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Dried Raspberry | 3.00 |
| 22 | Soy Crisps, 80% protein | 4.60 |
| | Total | 100.00 |

A. Chocolate Peanut Butter
Formula # 5367-45-13
Serving size 55 g, no calcium, 5 g alginate

| # | B. Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

C. Chocolate Peanut Butter
Formula # 5367-45-14
Serving size 55 g, no calcium, 5 g alginate
LBA alginate

| # | D. Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

Aw = 0.383

Chocolate Peanut Butter
Formula # 5367-45-15
E. Serving size 55 g, no calcium, 5 g alginate
GHB alginate

| # | F. Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-16
Serving size 55 g, no calcium, 5 g alginate (LBA alginate)
IV.

| # | A. Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-17
Serving size 55 g, no calcium
B. Spray Dry Batch #1

| # | C. Ingredient | Percentage |
|---|---|---|
| 1 | Sugar-Free Choc Coating | 20.00 |
| 2 | Corn Syrup | 17.00 |
| 3 | Glycerine | 3.00 |
| 4 | Peanut Butter | 10.00 |
| 5 | Peanut Butter Flavor | 2.00 |
| 6 | Maltodextrin | 0.00 |
| 7 | Erythritol | 4.00 |
| 8 | Alginate | 21.81 |
| 9 | Pectin | 0.00 |
| 10 | Peanut Flour | 3.40 |
| 11 | Tricalcium Phosphate | 0.00 |
| 12 | Soy Protein Isolate | 3.50 |
| 13 | Calcium Caseinate | 3.50 |
| 14 | Whey Protein Isolate | 3.50 |
| 15 | Peanuts | 8.29 |
| 16 | Soy Crisps | 0.00 |
| | Total | 100.00 |

Aw = 0.519

D. Chocolate Peanut Butter
Formula # 5367-45-18
Serving size 55 g, no calcium, 5 g alginate
LBA

| # | E. Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

F. Chocolate Peanut Butter
Formula # 5367-45-19
Serving size 55 g, no calcium
Spray Dry Batch # 2

| # | G. Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

H. Chocolate Peanut Butter
Formula # 5367-45-20
Serving size 55 g, no calcium
I. Spray Dry Batch # 3

| # | J. Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalcium Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

K. Chocolate Peanut Butter
Formula # 5367-45-21
Serving size 55 g, no calcium
Spray Dry Batch # 4

| # | L. Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.10 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 21.40 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 3.20 |
| 16 | Soy Crisps | 0.00 |
| | Total | 95.00 |

M. Chocolate Peanut Butter
Formula # 5367-45-22
Serving size 55 g, no calcium
Spray Dry Batch # 5

| # | N. Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalciurn Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

O. Chocolate Peanut Butter
Formula # 5367-45-23
Serving size 55 g, no calcium
Spray Dry Batch # 6

| # | P. Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.70 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 16.00 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 8.00 |
| 16 | Soy Crisps | 0.00 |
| | Total | 95.00 |

Q. Chocolate Peanut Butter
Formula # 5367-45-24
Serving size 55 g, no calcium
Spray Dry Batch #1

| # | R. Ingredient | Percentage |
|---|---|---|
| 1 | Sugar-Free Choc Coating | 20.00 |
| 2 | Corn Syrup | 17.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 21.81 |
| 10 | Pectin | 0.00 |
| 11 | Peanut Flour | 3.40 |
| 12 | Tricalcium Phosphate | 0.00 |
| 13 | Soy Protein Isolate | 1.00 |
| 14 | Calcium Caseinate | 1.00 |
| 15 | Whey Protein Isolate | 1.00 |
| 16 | Peanuts | 6.09 |
| 17 | Soy Crisps | 0.00 |
| | Total | 100.00 |

S. Chocolate Peanut Butter
Formula # 5367-45-25
Serving size 55 g, 5 g alginate
LBA (alginate)

| # | T. Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

V. Chocolate Peanut Butter
Formula # 5367-45-26
A. Serving size 55 g, 5 g alginate

| # | B. Ingredient | Percentage |
|---|---|---|
| 1 | Sugar-Free Choc Coating | 20.00 |
| 2 | Corn Syrup | 16.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 102.00 |

Chocolate Peanut Butter, Jan. 26, 2004
Formula # 5367-45-27
Serving size 55 g, 5 g alginate

| # | C. Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

VI. Chocolate Peanut Butter
Formula # 5367-45-30B, LBA
A. Serving size 55 g, 5 g alginate

| # | B. Ingredient | Percentage |
|---|---|---|
| 1 | Sugar-Free Choc Coating | 18.20 |
| 2 | HFCS | 10.00 |
| 3 | Glycerine | 7.00 |
| 4 | Maltitol | 7.00 |
| 5 | Peanut Butter | 3.00 |
| 6 | Peanut Butter Flavor | 3.00 |
| 7 | Vanilla | 0.50 |
| 8 | Sucrolose | 0.01 |
| 9 | Inulin | 5.00 |
| 10 | Erythritol | 8.00 |
| 11 | Alginate | 9.10 |
| 12 | Peanut Flour | 5.00 |
| 13 | Hydrolyzed Whey Isolate | 7.00 |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Total | 100.00 |
| | Aw = 0.402 | |

VII. Chocolate Peanut Butter
Formula # 5367-45-28
Serving size 55 g, 5 g alginate

| # | A. Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 0.00 |
| 8 | Inulin | 0.00 |
| 9 | Erythritol | 5.00 |
| 10 | Alginate | 0.00 |
| 11 | Pectin | 0.00 |
| 12 | Spray Dry Alginate | 14.26 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

VIII. Chocolate Peanut Butter
Formula # 5367-45-29
Serving size 55 g, 5 g alginate

| # | A. Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 4.76 |
| 8 | Inulin | 0.00 |
| 9 | Erythritol | 5.00 |
| 10 | Alginate | 0.00 |
| 11 | Pectin | 0.00 |
| 12 | Spray Dry Alginate | 9.50 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

IX. Chocolate Peanut Butter
Formula # 5367-45-34
Serving size 55 g, 5 g alginate

| # | A. Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

X. Chocolate Peanut Butter
Formula # 5367-45-35
Serving size 55 g, 3 g alginate

| # | A. Ingredient | Percentage |
|---|---|---|
| 1 | Sugar-Free Choc Coating | 20.00 |
| 2 | High Fructose Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| 7 | Inulin | 5.00 |
| 8 | Erythritol | 5.00 |
| 9 | Alginate | 5.45 |
| 10 | Peanut Flour | 3.40 |
| 11 | Dicalcium Phosphate Anyhdrous | 1.88 |
| 12 | Soy Protein Isolate | 4.00 |
| 13 | Calcium Caseinate | 4.00 |
| 14 | Whey Protein Isolate | 4.00 |
| 15 | Peanuts | 9.27 |
| | Total | 100.00 |

B. Chocolate Peanut Butter
Formula # 5367-45-01
Serving size 55 g, contains 200 mg Ca, 1 g alginate, 1 g pectin, 3 g inulin

| # | C. Ingredient | Percentage |
|---|---|---|
| 1 | Sugar-Free Choc Coating | 18.00 |
| 2 | Water | 12.50 |
| 3 | Glycerine | 9.00 |
| 4 | Peanut Butter | 10.00 |
| 8 | Peanut Butter Flavor | 1.00 |
| 11 | Inulin | 5.45 |
| 12 | Erythritol | 8.00 |
| 13 | Fructose | 3.50 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Peanut Flour | 7.40 |
| | Total | 100.00 |
| | Aw 0.686 | |

XI. Chocolate Peanut Butter
Formula # 5367-45-02
Serving size 55 g, contains 200 mg Ca, 1 g alginate, 1 g pectin, 3 g inulin

| # | A. Ingredient | Percentage |
|---|---|---|
| 1 | Sugar-Free Choc Coating | 15.00 |
| 2 | Water | 14.00 |
| 3 | Glycerine | 10.00 |
| 4 | Peanut Butter | 10.00 |
| 8 | Peanut Butter Flavor | 2.00 |
| 11 | Inulin | 5.45 |
| 12 | Erythritol | 7.00 |
| 15 | Alginate | 2.15 |
| 16 | Pectin | 3.04 |
| 21 | Peanut Flour | 5.00 |
| 20 | Tricalcium Phosphate | 0.96 |
| 17 | Soy Protein Isolate | 5.00 |
| 18 | Calcium Caseinate | 5.00 |
| 19 | Whey Protein Isolate | 7.50 |
| | Peanuts | 5.00 |
| | Soy Crisps | 2.90 |
| | Total | 100.00 |
| | Aw 0.726 | |

XII. Chocolate Peanut Butter
Formula # 5367-45-03
Serving size 55 g, contains 200 mg Ca, 1 g alginate, 1 g pectin, 3 g inulin

| # | A. Ingredient | Percentage |
|---|---|---|
| 1 | Sugar-Free Choc Coating | 15.00 |
| 2 | Water | 14.50 |
| 3 | Glycerine | 11.00 |
| 4 | Peanut Butter | 10.00 |
| 8 | Peanut Butter Flavor | 2.00 |
| 11 | Inulin | 5.45 |
| 12 | Erythritol | 10.00 |
| 15 | Alginate | 2.15 |
| 16 | Pectin | 3.04 |
| 21 | Peanut Flour | 3.40 |
| 20 | Tricalcium Phosphate | 0.96 |
| 17 | Soy Protein Isolate | 3.50 |
| 18 | Calcium Caseinate | 3.50 |
| 19 | Whey Protein Isolate | 3.50 |
| | Hydrolysed Whey | 3.50 |
| | Peanuts | 6.00 |
| | Soy Crisps | 2.50 |
| | Total | 100.00 |
| | A w 0.710 | |

B. Chocolate Raspberry
Formula # 5367-44-01
Serving size 45 g, contains 200 mg Ca, 5% alginate/pectin mix, 3 g inulin

| # | C. Ingredient | Percentage |
|---|---|---|
| 1 | Chocolate Coating | 15.00 |
| 2 | HFCS | 10.00 |
| 3 | Water | 10.00 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.20 |
| 11 | Inulin | 6.70 |
| 12 | Erythritol | 5.00 |
| 13 | Malic Acid | 0.25 |
| 14 | Tricalcium Phosphate | 1.15 |
| 15 | Alginate Pectin Mix | 5.00 |
| 16 | Soy Protein Isolate | 7.00 |
| 17 | Calcium Caseinate | 6.00 |
| 18 | Whey Protein Isolate | 6.00 |
| 19 | Dried Raspberry | 4.85 |
| 20 | Soy Crisps, 80% protein | 5.00 |
| | Total | 100.00 |
| | Aw at 0.677 | |

XIII. Chocolate Peanut Butter
A. Formula # 5367-45-04
Serving size 55 g, contains 200 mg Ca, 1 g alginate, 1 g pectin, 3 g inulin

| # | B. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 2.15 |
| 9 | Pectin | 3.04 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 100.00 |
| | Aw 0.698 | |

XIV. Chocolate Raspberry
Formula # 5367-44-02
Serving size 45 g, contains 200 mg Ca, 5% alginate/pectin mix, 3 g inulin

| # | A. Ingredient | Percentage |
|---|---|---|
| 1 | Chocolate Coating | 15.00 |
| 2 | HFCS | 10.50 |
| 3 | Water | 10.50 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.50 |
| 11 | Inulin | 6.70 |
| 12 | Erythritol | 2.00 |
| 13 | Malic Acid | 0.25 |
| 14 | Alginate | 2.50 |
| 15 | Pectin | 2.50 |
| 16 | Soy Protein Isolate | 7.00 |
| 17 | Calcium Caseinate | 6.00 |
| 18 | Whey Protein Isolate | 6.00 |
| 19 | Tricalcium Phosphate | 1.15 |
| 20 | Dried Raspberry | 6.00 |
| 21 | Soy Crisps, 80% protein | 5.55 |
| | Total | 100.00 |

XV. Chocolate Peanut Butter
Formula # 5367-45-31
Serving size 55 g, 5 g alginate

| # | A. Ingredient | Percentage |
|---|---|---|
| 1 | Sugar-Free Choc Coating | 18.20 |
| 2 | HFCS | 12.00 |
| 3 | Maltitol | 12.00 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.01 |
| | Mix for 0.5 min | |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Mix for 1 min | |
| | Total | 100.00 |
| | Aw = 0.52 | |

XVI. Chocolate Peanut Butter
Formula # 5367-45-05
Serving size 55 g, contains 200 mg Ca, 1 g alginate, 1 g pectin, 3 g inulin
GHB

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 4.30 |
| 9 | Pectin | 0.00 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 99.11 |
| | Aw 0.713 | |

XVII. Chocolate Peanut Butter
Formula # 5367-45-06
Serving size 55 g, contains 200 mg Ca, 1 g alginate, 1 g pectin, 3 g inulin

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 7.00 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | MonoCalcium Phosphate | 4.60 |
| | Total | 100.00 |
| | a = 0.705 | |

XVIII. Chocolate Peanut Butter
Formula # 5367-45-07
Serving size 55 g, contains 200 mg Ca, 1 g alginate, 1 g pectin, 3 g inulin

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 6.10 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 5.50 |
| | Total | 100.00 |
| | Aw 0.690 | |

XIX. Chocolate Peanut Butter, Dec. 22, 2003
Formula # 5367-45-08
Serving size 55 g, contains 300 mg Ca, 1 g alginate, 1 g pectin, 3 g inulin

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 2.15 |
| 9 | Pectin | 3.04 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

XX. Chocolate Peanut Butter
Formula # 5367-45-09
Serving size 55 g, contains 300 mg Ca, 3 g inulin

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 5.52 |
| 8 | Alginate | 0.00 |
| 9 | Pectin | 0.00 |
| 10 | Peanut Flour | 8.59 |
| 11 | Tricalcium Phosphate | 1.44 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| 18 | Calcium Lactate | 0.00 |
| | Total | 100.00 |

B. Chocolate Peanut Butter
Formula # 5367-45-10
Serving size 55 g, contains 200 mg Ca, 3 g inulin
Test layer bar

| # | C. Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 10.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 100.00 |

XXI. Chocolate Peanut Butter
Formula # 5367-45-11
Serving size 55 g
Test layer bar

| # | A. Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 0.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 90.00 |

XXII. Chocolate Peanut Butter
Formula # 5367-45-12
Serving size 55 g, contains 300 mg Ca

| # | A. Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 5.19 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

4. Beverage Formulations

A variety of beverage formulations were prepared having the following formulations:

Raspberry Beverage 5877-13

| Ingredient | % Formula |
|---|---|
| Water | 85.333 |
| Trehalose | 3.0 |
| Sodium Hexametaphosphate | 0.100 |
| EDTA | 0.003 |
| Manugel GHB | 0.450 |
| Pectin USP-L200, CP Kelco | 0.850 |
| Manugel LBA | 0.400 |
| Sodium Citrate Anh. | 0.100 |
| Sucralose, 25% Sol'n | 0.018 |
| Juice Conc. Raspberry 65 Brix | 2.000 |
| Juice Conc. Apple 70 Brix | 7.500 |
| Flvr. Blackberry 596447 | 0.089 |
| Givaudan | |

Pina Colada 5587-35

| Ingredient | % Formula |
|---|---|
| Water | 92.351 |
| Trehalose | 3.0 |
| Inulin F97 | 0.86 |
| GHB Alginate | 0.625 |
| TIC Rapid Set Pectin | 0.625 |
| Sodium Citrate Dihydrate | 0.05 |
| Isomaltulose | 2.0 |
| Sucralose, 25% Sol'n | 0.016 |
| Pina Colada Flv FADR352, | 0.233 |
| WILD | |
| White Dispersion | 0.14 |
| Malic Acid, 25% Sol'n | 0.1 |

Tropical

| Ingredient | % Formula |
|---|---|
| Water | 94.571 |
| Apple JC, 70 brix | 2.3 |
| Manugel LBA | 0.9 |
| Manugel GHB | 0.15 |
| Pectin, USP-L200 | 0.65 |
| Malic Acid, 25% Sol'n | 0.85 |
| Sodium Citrate, Dihydrate | 0.25 |
| EDTA | 0.015 |
| Sucralose, 25% Sol'n | 0.043 |
| Kiwi Flv, Ottens #10301 | 0.09 |
| Passionfruit Flv, Robertet NV-18, | 0.1 |
| 586 | |
| Pineapple Flv, WFF #03CF770 | 0.06 |
| Yellow #5, 1% Sol'n | 0.016 |
| Yellow #6, 1% Sol'n | 0.005 |

Key Lime

| Ingredient | % Formula |
|---|---|
| Water | 94.236 |
| Apple JC, 70 brix | 2.3 |
| Manugel LBA | 0.9 |
| Manugel GHB | 0.15 |
| Pectin, USP-L200 | 0.65 |
| Malic Acid, 25% Sol'n | 0.95 |
| Sodium Citrate, Dihydrate | 0.25 |
| EDTA | 0.015 |
| Sucralose, 25% Sol'n | 0.043 |
| Lime Flv, Sensient #2014815 | 0.29 |
| Passionfruit Flv, Robertet NV-18, 586 | 0.04 |
| Creamy Shake Flv, Sensient #2014816 | 0.15 |
| Yellow #5, 1% Sol'n | 0.011 |
| Blue #1, 0.1% Sol'n | 0.015 |

Key Lime 5587

| Ingredient | % Formula |
|---|---|
| Water | 95.117 |
| Apple JC, 70 brix | 2.3 |
| Manugel LBA | 0.9 |
| Manugel GHB | 0.15 |
| Pectin, USP-L200 | 0.65 |
| Malic Acid | 0.288 |
| Sodium Citrate, Dihydrate | 0.25 |
| EDTA | 0.002 |
| Sucralose, 25% Sol'n | 0.011 |
| Lime Flv, Sensient #2014815 | 0.29 |
| Passionfruit Flv, Robertet NV-18, 586 | 0.04 |
| Yellow #5, 10% Sol'n | 0.001 |
| Blue #1, 1% Sol'n | 0.0015 |

Key Lime 1287

| Ingredient | % Formula |
|---|---|
| Water | 95.946 |
| Apple JC, 70 brix | 2.3 |
| Manugel LBA | 0.430 |
| Manugel GHB | 0.125 |
| Pectin, USP-L200 | 0.400 |
| Malic Acid | 0.204 |
| Sodium Citrate, Dihydrate | 0.25 |
| EDTA | 0.002 |
| Sucralose, 25% Sol'n | 0.011 |
| Lime Flv, Sensient #2014815 | 0.290 |
| Passionfruit Flv, Robertet NV-18, 586 | 0.040 |
| Yellow #5, 10% Sol'n | 0.001 |
| Blue #1, 1% Sol'n | 0.0015 |

Key Lime 2901

| Ingredient | % Formula |
|---|---|
| Water | 96.533 |
| Apple JC, 70 brix | 2.3 |
| Manugel LBA | 0.170 |
| Manugel GHB | 0.050 |
| Pectin, USP-L200 | 0.160 |
| Malic Acid | 0.192 |
| Sodium Citrate, Dihydrate | 0.25 |
| EDTA | 0.002 |
| Sucralose, 25% Sol'n | 0.011 |
| Lime Flv, Sensient #2014815 | 0.290 |
| Passionfruit Flv, Robertet NV-18, 586 | 0.040 |
| Yellow #5, 10% Sol'n | 0.001 |
| Blue #1, 1% Sol'n | 0.0015 |

Key Lime 4174 (control - no fiber)

| Ingredient | % Formula |
|---|---|
| Water | 97.032 |
| Apple JC, 70 brix | 2.3 |
| Manugel LBA | 0.000 |
| Manugel GHB | 0.000 |
| Pectin, USP-L200 | 0.000 |
| Malic Acid | 0.172 |
| Sodium Citrate, Dihydrate | 0.25 |
| EDTA | 0.002 |
| Sucralose, 25% Sol'n | 0.011 |
| Lime Flv, Sensient #2014815 | 0.203 |
| Passionfruit Flv, Robertet NV-18, 586 | 0.028 |
| Yellow #5, 10% Sol'n | 0.001 |
| Blue #1, 1% Sol'n | 0.0015 |

Peachy Grapefruit

| Ingredient | % Formula |
|---|---|
| Water | 94.465 |
| Apple JC, 70 brix | 2.3 |
| Manugel LBA | 0.9 |
| Manugel GHB | 0.15 |
| Pectin, USP-L200 | 0.65 |
| Malic Acid, 25% Sol'n | 0.85 |
| Sodium Citrate, Dihydrate | 0.25 |
| EDTA | 0.0015 |
| Sucralose, 25% Sol'n | 0.043 |
| Peach Grapefruit Flv, Robertet NV-19, 851 | 0.25 |
| Pink Grapefruit Flv, Givaudan #553342 | 0.03 |
| Peach Flv, David Michael #20843 | 0.10 |
| Red 40, 1% Sol'n | 0.01 |

Peachy Grapefruit 7910

| Ingredient | % Formula |
|---|---|
| Water | 95.123 |
| Apple JC, 70 brix | 2.30 |
| Manugel LBA | 0.9 |
| Manugel GHB | 0.15 |
| Pectin, USP-L200 | 0.65 |
| Malic Acid, granular | 0.234 |
| Sodium Citrate, Dihydrate | 0.25 |
| EDTA | 0.002 |
| Sucralose, 25% Sol'n | 0.011 |
| Peach Grapefruit Flv, Robertet NV-19, 851 | 0.25 |
| Pink Grapefruit Flv, Givaudan #553342 | 0.03 |
| Peach Flv, David Michael #20843 | 0.10 |
| Red 40, 10% Sol'n | 0.001 |

Peachy Grapefruit 8211

| Ingredient | % Formula |
|---|---|
| Water | 95.912 |
| Apple JC, 70 brix | 2.30 |
| Manugel LBA | 0.430 |
| Manugel GHB | 0.125 |
| Pectin, USP-L200 | 0.400 |
| Malic Acid, granular | 0.190 |
| Sodium Citrate, Dihydrate | 0.25 |
| EDTA | 0.002 |
| Sucralose, 25% Sol'n | 0.011 |
| Peach Grapefruit Flv, Robertet NV-19, 851 | 0.25 |
| Pink Grapefruit Flv, Givaudan #553342 | 0.03 |
| Peach Flv, David Michael #20843 | 0.10 |
| Red 40, 10% Sol'n | 0.001 |

Peachy Grapefruit 1451

| Ingredient | % Formula |
|---|---|
| Water | 96.502 |
| Apple JC, 70 brix | 2.30 |
| Manugel LBA | 0.170 |
| Manugel GHB | 0.050 |
| Pectin, USP-L200 | 0.160 |
| Malic Acid, granular | 0.175 |
| Sodium Citrate, Dihydrate | 0.25 |
| EDTA | 0.002 |
| Sucralose, 25% Sol'n | 0.011 |
| Peach Grapefruit Flv, Robertet NV-19, 851 | 0.25 |
| Pink Grapefruit Flv, Givaudan #553342 | 0.03 |
| Peach Flv, David Michael #20843 | 0.10 |
| Red 40, 10% Sol'n | 0.001 |

Peachy Grapefruit 2402 (control - no fiber)

| Ingredient | % Formula |
|---|---|
| Water | 97.006 |
| Apple JC, 70 brix | 2.30 |
| Manugel LBA | 0.00 |
| Manugel GHB | 0.00 |
| Pectin, USP-L200 | 0.00 |
| Malic Acid, granular | 0.165 |
| Sodium Citrate, Dihydrate | 0.25 |
| EDTA | 0.002 |
| Sucralose, 25% Sol'n | 0.011 |
| Peach Grapefruit Flv, Robertet NV-19, 851 | 0.175 |
| Pink Grapefruit Flv, Givaudan #553342 | 0.021 |
| Peach Flv, David Michael #20843 | 0.070 |
| Red 40, 10% Sol'n | 0.001 |

5. Pig Studies of Beverage Formulations

Trial 1: Analysis of pH and Viscosity of Pig Digesta after Consumption of Liguid Ingestible Compositions

Animals: The experiment was performed at the Cargill Animal Nutrition Innovation Center in Elk River, Minn. Starter pigs weighing 20 to 25 Kg were used. Animals were weaned for approximately 4 weeks and fed standard PorkTrack 25-50 animal feed.

Acclimation: Animals were acclimated to an experimental beverage that did not contain any anionic soluble fiber sources and limit-fed for two weeks prior to the start of the experiment. Each experimental fiber-fed pig was fed a raspberry flavored anionic-soluble fiber containing drink (˜500 ml) (see below) to encourage complete consumption in as short of a time span as possible. Placebo fed pigs were fed a raspberry-flavored version that did not contain any anionic soluble fiber. Water and limited feed were offered for the remainder of the experimental period after consumption of the beverage.

Fiber-containing Raspberry Beverage Formula (5877-13)

| Ingredient | Percent Formula |
|---|---|
| Water | 85.333 |
| Sodium Citrate, Anhydrous | 0.100 |
| Sweetener 25% sucralose | 0.018 |
| Trehalose, Ascend | 3.000 |
| Juice Concentrate, Raspberry, 65 Brix | 2.000 |
| Juice Concentrate, Apple, 70 Brix | 7.500 |
| Manugel GHB, Sodium Alginate | 0.450 |
| Pectin (citrus) Type USP-L200, CP Kelco | 0.850 |
| Manugel LBA, Sodium Alginate | 0.400 |
| Sodium Hexametaphosphate | 0.100 |
| EDTA, Versene CA, DOW Chem | 0.003 |
| Natural Raspberry WONF-Symrise (813750) | 0.157 |
| Flvr, Blackberry, 596447, Givaudan | 0.089 |

Experimental design: Water was restricted for 16 hours prior to the start of the experiment. Blood samples (7 ml clot tube) from each animal were taken prior to offering the raspberry drink. Approximately 230 mls of experimental (see above) or control beverage (without alginate and pectin anionic soluble fibers) were fed to each animal. Another blood sample (7 ml clot tube) was drawn just prior to euthanasia of each animal.

One animal from the experimental group and one animal from the placebo control group were euthanized at 10, 30, 60, 90, 120, and 180 minutes post drink consumption. As quickly as possible, animals were surgically opened, and the stomach and duodenum were clamped at both ends. The stomach and duodenum were removed, and their contents emptied into a labeled container on ice.

Analysis: Viscosity and pH of intestinal contents were measured immediately. Viscosity was determined using an LVDVII+ viscometer with cone and plate attachment, and an RVDVII+ with a ULV attachment, or using a qualitative scale (see below). Blood samples were used to measure blood glucose at an independent laboratory (Marshfield Veterinary Diagnostics Laboratory, Marshfield, Wis.). Digesta pH values were recorded from a standart laboratory pH meter.

Results and Discussion

Animals were healthy at the start of both the acclimation and experimental periods. Beverages were readily ingested by all animals, with most consuming the given amount in under a minute. Visual observations of the stomach fluid were recorded, and qualitative viscosity measurements were performed, using a 0 to 4 scale (where 0=no gel particles and 4=plentiful gel particles). Stomach contents from animals fed fiber-containing beverages contailed gel particles in the digesta within 10 minutes after ingestion (see below). These stomach contents showed a very heterogeneous gel of hard gel particles. The stomach lining near the pyloric valve showed a visible gel on the surface that persisted until 120 minutes after beverage ingestion. There appeared to be no adverse reactions of the stomach and intestines from ingestion of an anionic soluble fiber-containing beverage.

Visual Qualification of Gel Formation in the Stomach.

| Time after | | Anionic |
|---|---|---|
| Ingestion | | Soluble Fiber- |
| (min) | Placebo* | containing* |
| 10 | 0 | 1 |
| 30 | 0 | 4 |
| 60 | 0 | 4 |
| 90 | 0 | 3 |
| 120 | 0 | 2 |
| 180 | 0 | 0 |

The stomach pH did not decline much in samples measured 10 minutes after beverage consumption. By 30 minutes, the stomach pH was 1.85. There were only minor differences in stomach pH between placebo and fiber-fed animals. The most significant difference was observed at 120 minutes after ingestion. The placebo animal's stomach had a pH of 5.78, and the fiber-fed animal had a pH of 3.62. Visual observation indicated the placebo animal had bile salts present in the stomach chyme. The trend reversed itself in the 180-minute samples. Stomach contents of the fiber-fed pig had a pH of 6.87, and the placebo pig had a stomach pH of 2.23. Visual observation of chyme indicated bile salts were present in the fiber-fed animal, but placebo chyme appeared more digested. This may be an indication that stomach-emptying rates may have been slowed by the presence of fiber (pectin and alginate) in the beverage.

An additional experiment was done where single animals were fed either a placebo or a fiber-containing beverage (8 oz), followed immediately by feeding of an aliquot of calcium lactate (300 mg in 8 oz). Animals were sacrificed 45 minutes after beverage consumption, and the stomach and duodenum were removed. Stomach fluid of fiber-fed animals contained a larger amount of gel particles than observed in the earlier experiment. The gel particles also seemed to be firmer. The stomach lining had an appreciable amount of gel on its surface. The animal fed calcium and fiber had a stomach pH of 2.99, and the placebo animal had a stomach pH of 2.27.

Results from this study confirmed our in vitro stomach model results that a beverage containing alginate and pectin anionic soluble fiber would form a gel in the stomach when ingested by a live animal.

Trial 2: Evaluation of Adverse Effects of Ingestion of Liquid Ingestible Compositions

This research trial was designed to run for two weeks to observe any adverse effects on pigs from consuming an alginate:pectin anionic soluble fiber-containing beverage.

Animals: The experiment was done at the Cargill Animal Nutrition Innovation Center in Elk River, Minn. Starter pigs of mixed sex weighing 28.8 Kg were used in the study. Animals were weaned for approximately 5 weeks and fed standard PorkTrack 25-50 animal feed.

Experimental Design: Animals were not acclimated to the beverage used in these experiments. On day 0 of the feeding trial, animals were weighed, body temperatures recorded, offered feed (PT 25-50) was weighed, and blood samples (˜5 ml) were drawn into a clot tube and (˜5 ml) into an EDTA tube. Ten animals were fed 250 ml of an experimental beverage, ten animals were fed 250 ml of a placebo beverage, and 10 animals received no beverage. The beverage at room temperature was fed ad libitum in the morning and evening for 14 consecutive days.

Animals were observed daily at approximately 0800 hours for any adverse effects. Daily skin temperatures and fecal scores were recorded. Rectal temperatures were measured on each Monday, Wednesday, and Friday. Skin temperature was taken using an infrared thermometer; rectal temperatures were measured with a digital thermometer with a metal probe. Fecal scores were on a scale of 0 (normal) to 4 (severe, bloody diarrhea). On day 14, body weights, and feed weights were recorded and blood samples (˜5 ml) were drawn into a clot tube and (˜5 ml) into an EDTA tube.

Each experimental pig was offered an anionic soluble fiber-containing raspberry flavored drink (˜250 ml) (see below) to encourage complete consumption in as short of a time-span as possible. Placebo pigs received a similar beverage that lacked anionic soluble fiber. Water and feed were offered for the remainder of the experimental period after consumption of the beverage.

Raspberry beverage formula (5877-13)

| | |
|---|---|
| Water | 85.333 |
| Sodium Citrate, Anhydrous | 0.100 |
| Sweetener 25% sucralose | 0.018 |
| Trehalose, Ascend | 3.000 |
| Juice Concentrate, Raspberry, 65 Brix | 2.000 |
| Juice Concentrate, Apple, 70 Brix | 7.500 |
| Manugel GHB, Sodium Alginate | 0.450 |
| Pectin (citrus) Type USP-L200, CP | 0.850 |
| Kelco | |
| Manugel LBA, Sodium Alginate | 0.400 |
| Sodium Hexametaphosphate | 0.100 |
| EDTA, Versene CA, DOW Chem | 0.003 |
| Natural Raspberry WONF-Symrise | 0.157 |
| (813750) | |
| Flvr, Blackberry, 596447, Givaudan | 0.089 |

Animals were healthy at the start of the experiment. Beverages were readily ingested by all animals. No visible adverse reactions were observed during the experimental period. Skin and rectal temperatures were normal in all animals. Fecal scores remained normal. Blood chemistries for all animals were in the normal ranges. The results overall indicated that there were no adverse effects to hepatic or renal systems in a short-term exposure to alginate:pectin anionic soluble fiber-containing beverages when ingested by young pigs.

Trial 3: Effect of Calcium on Digesta Viscosity

Animals: The experiment was done at the Cargill Animal Nutrition Innovation Center in Elk River, Minn. Starter pigs of mixed sex weighing 19.5 to 30.5 Kg were used in this study. Animals were weaned for approximately 5 weeks and fed standard PorkTrack 25-50 animal feed.

Acclimation: Animals were acclimated to an experimental beverage that did not contain anionic soluble fiber and fed ad libitum for two weeks prior to the start of the experiment. Each fiber-fed pig was fed an anionic-soluble fiber-containing raspberry flavored drink (˜500 ml) (see below) to encourage complete consumption in as short of a time span as possible. Placebo fed pigs were fed a similar beverage that lacked anionic soluble fiber. Water and feed were offered for the remainder of the experimental period after consumption of the beverage.

Raspberry beverage formula (5877-13)

| Ingredient | Percent Formula |
|---|---|
| Water | 85.333 |
| Sodium Citrate, Anhydrous | 0.100 |
| Sweetener 25% sucralose | 0.018 |
| Trehalose, Ascend | 3.000 |
| Juice Concentrate, Raspberry, 65 Brix | 2.000 |
| Juice Concentrate, Apple, 70 Brix | 7.500 |
| Manugel GHB, Sodium Alginate | 0.450 |
| Pectin (citrus) Type USP-L200, CP Kelco | 0.850 |
| Manugel LBA, Sodium Alginate | 0.400 |
| Sodium Hexametaphosphate | 0.100 |
| EDTA, Versene CA, DOW Chem | 0.003 |
| Natural Raspberry WONF-Symrise (813750) | 0.157 |
| Flvr, Blackberry, 596447, Givaudan | 0.089 |

Experimental design: Water was restricted for 2.75 hours prior to the start of the experiment. Gel 1 pigs were fed approximately 250 mls of experimental (see above) soluble anionic fiber-containing beverage, as described previously. Gel 2 pigs were fed the experimental beverage in a similar manner. Immediately after consumption, however, Gel 2 animals were fed 125 ml of calcium lactate (2.4 mg/ml). A single animal was fed a placebo beverage (without fiber).

One animal from each experimental group (Gel 1 and Gel 2) was euthanized at 10, 45, 90, 150, 210, and 250 minutes post drink consumption. The placebo animal was sacrificed 10 minutes after drink consumption. As quickly as possible, animals were surgically opened, and the stomach and duodenum were clamped at both ends. The stomach and duodenum were removed, and their contents emptied into a labeled container on ice.

Analysis: Viscosity and pH of stomach and small intestinal contents were measured immediately. Stomach digesta viscosity was determined using a modified Bostwick consistometer. Small intestinal digesta viscosity was measured using a Brookfield Viscometer with a vane spindle. Viscosity readings were taken after applying shear stress for 15 seconds. Digesta pH values were recorded from a standard laboratory pH meter.

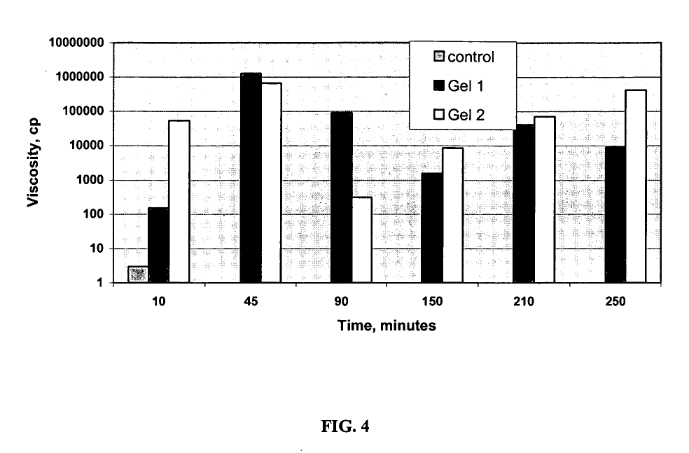

Animals were healthy at the start of both the acclimation and experimental periods. Beverages were readily ingested by all animals. As observed in the Trial 1 experiments above, stomach contents from animals fed anionic soluble fiber-containing beverages contained gel particles that increased the viscosity of the digesta within 10 minutes after ingestion. Stomach contents showed a very heterogeneous gel of hard gel particles interspersed in the digestive contents. The stomach lining near the pyloric valve showed a visible gel in the 10 and 45 minute pigs, but this gel was more difficult to find in later time periods because of the propensity of animal feed to cling to the intestinal walls. In the 10-minute pigs, anionic soluble fiber-containing beverages supplemented with calcium (Gel 2) formed digesta with greater viscosity than the fiber beverage alone, but the 45 and 90-minute digesta samples from Gel 1 pigs had greater viscosity. This trend reversed itself in the later samples with Gel 2 animals having slightly greater viscosity than Gel 1 animals. It is possible calcium from the animal feed may have contributed to results observed in the first 90 minutes with the Gel 1 animals. Pig starter feed contains significant amounts of calcium for the young animal. See FIG. 4.

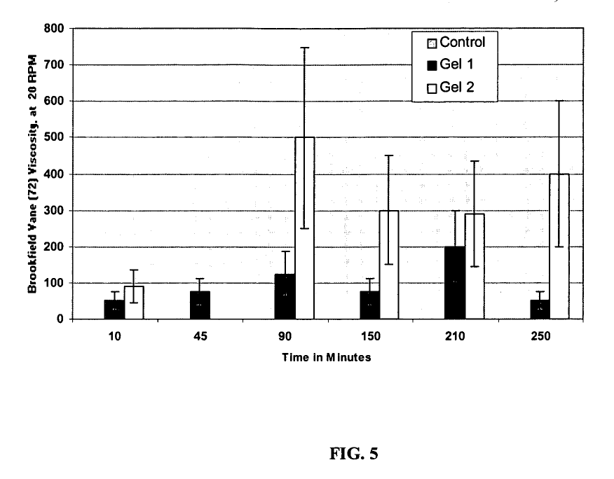

Duodenal fluids were viscous upon visual observation, resembling mucous. Duodenal fluid viscosity was measured with a Brookfield viscometer using a vane spindle. Viscosity readings were recorded after 15 seconds because these gels were shear sensitive, and longer shear stress tended to disrupt the gels and give erroneous results. Gel in the stomach had a tendency to be small spheres of gel approximately 0.1 to 0.5 cm in diameter, and were dispersed throughout the digestive contents. See FIG. 5.

There appeared to be no adverse reactions of the stomach and intestinal lining from ingestion the anionic soluble fiber-containing beverage. Stomach and small intestinal pH was buffered by presence of feed in the GI tract of all animals. There were only minor differences in pH values of the stomach and small intestine regardless of treatment. Feed intake was monitored in all animals. Gel 2 animals, with the exception of the 150-minute pig, had lower postprandial intakes than Gel 1 pigs.

Results from this study re-confirmed earlier experiments that showed a beverage containing alginate and pectin fibers would form a gel in the stomach when ingested by a live animal. In addition, the presence of calcium increased digesta viscosity greater than the fiber alone. This experiment also showed gel integrity is maintained as digesta enters into upper regions (duodenum) of the small intestine. In conclusion, beverages containing alginate and pectin fibers will gel in the stomach's acidic environment after ingestion. Additional consumption of calcium lactate will form a firmer gel in the stomach that maintains integrity into the small intestine.

6. Calcium Alginate Microparticle and Nanoparticle Preparation

Materials

Refined, bleached and deodorized corn and soybean oil were purchased locally for use in the microemulsion formulations. Mazol 80 (food grade, mixture of ethoxylated mono and diglycerides) was obtained from BASF (Mt. Olive, N.J.). Tween 60, Tween 80, and SPAN 80 were supplied by TCI America (Portland, Oreg.). Sucrose stearate S-1570 was obtained from Mitsubishi Chemical (White Plains, N.J.). Triton X-100, 1-butanol, cyclohexane and hexanol were obtained from VWR. Octanoic acid was provided by Sigma Aldrich (Milwaukee, Wis.). The following sodium alginates were used: Keltone HV and Manugel LBA, provided by ISP (Wayne, N.J.). The surfactants listed above are all nonionic emulsifiers.

Macroemulsion Technology

A standard emulsification technique reported by Wan et al. (Wan, L.S.C., Heng, P. W. S., and Chan, L. W., J Microencapsulation 9 (1992) 309) was used to produce microparticles of calcium alginate. Fifty grams of a 2 wt % aqueous solution of sodium alginate was dispersed in 75 g of isooctane containing SPAN 80 (2 wt %) at 1000 rpm for 10 min. After the emulsion was prepared, another 5 g of water containing 2 wt % of Tween 80 was added after which 20 g of a 5 wt % CaCl₂ solution was added to react with the alginate. The microspheres were centrifuged and the oil/water emulsion decanted. Average particle size was determined by light microscopy. The size ranged from 5-15 μm.

1. Microemulsion Technology

The following standard procedure was used to prepare food and non-food grade microemulsions. Microemulsion solutions were prepared based upon phase diagram recommendations outlined in methods from multiple journal articles (Parris, N., Joubran, R. F., and Lu, D. P., J Agric. Food Chem. 42 (1994) 1295; Glatter, O. et al., J Coll Interface. Sci. 241 (2001) 215; Spernath, A. et al., J Agric. Food Chem. 50 (2002) 6917; Shen, M., Guo, R., J Surf and Det. 6 (2003) 265; and Wan, L. S. C., Heng, P. W. S., and Chan, L. W., J Microencapsulation 9 (1992) 309).

The solutions were prepared in small glass vials with all calculations based on a 5-25 mL working volume. A 2 wt % alginate solution was added drop-wise until the solution no longer turned clear. All solutions were slowly stirred and slightly heated (45 to 55° C.). The initial mass of the vial plus solution compared to the final mass of the vial plus alginate solution was used to determine the percentage of microemulsion produced. After combining the two microemulsions together, the calcium alginate particles were recovered by decanting the oil, centrifugation and washing with water/isopropanol (5/95 vol %). Average particle size was determined by a Brookhaven particle size analyzer (dynamic light scattering) and Transmission Electron Microscopy (TEM).

Food and non-food grade surfactant systems were evaluated. The food grade system (corn oil, Mazol 80 and ethanol) microemulsions demonstrated a bimodal particle distribution, with the large particles averaging 75 nm and the small particles averaging 11 nm in diameter.

The non-food grade system (TX-100, hexanol, and cyclohexane) microemulsions demonstrated web-like structures, with nanoparticles suspended inside and outside the web. Small, dendritic protrusions extended from each particle, forming a weak web. The average particle diameter was 85 nm.

2. Materials

Tween 80 and SPAN 80 were supplied by TCI America (Portland, Oreg.). The following sodium alginates were used: Keltone HV and Manugel LBA, provided by ISP (Wayne, N.J.). The surfactants listed above are all nonionic emulsifiers.

Macroemulsion Technology

Isooctane or hexane (350 g) was used as the oil phase. The following was added while stirring: 8 g surfactant (Span 80), 250 ml 5% Na Alginate (drop wise), 4 g Tween and 18 g 15% CaCl₂. After the calcium was added, the mixture was centrifuged and the oil phase decanted. The slurry was then dried by stripping off the hexane using a rotovap under slight vacuum. This process yields about 12-13 g of dry powder (˜100% yield).

Dry calcium alginate particles were prepared using the macroemulsification technique. A suspension of calcium alginate particles in a low pH beverage containing free sodium alginate showed early gelling, indicating calcium exchange. The stability of these particles in the presence and absence of sodium alginate for different buffered pH systems (pH 2-8) may be evaluated in the future.

7. Influence Of A Fiber-Blend On Blood Glucose Control In A Diabetic Patient

This brief report describes a small study with a diabetic patient to study blood glucose response after ingestion of an alginate:pectin beverage followed by a glucose challenge.

Subject: A 48 year-old Caucasian male with Type II diabetes (T2D) was used for this study. The patient had maintained reasonable blood glucose control for several months prior to the experiment, and had given an informed consent._{▬▬▬▬}

Test: The patient on two separate mornings consumed 12 ounces of a raspberry flavored beverage (see below) that contained an alginate:pectin fiber blend or a control (containing no fiber). Immediately after consuming the beverages, the patient consumed 4 ounces of glucose (75 g) solution. Blood samples were taken by finger prick every 25 minutes for 175 minutes. Blood glucose was determined using a OneTouch UltraSmart glucometer (LifeScan, Inc., Milpitas, Calif.).

Fiber Blend Beverage

| Ingredient | |
|---|---|
| Water | 85.333 |
| Sodium Citrate, Anhydrous | 0.100 |
| Sweetener 25% sucralose | 0.018 |
| Trehalose, Ascend | 3.000 |
| Juice Concentrate, Raspberry, 65 Brix | 2.000 |
| Juice Concentrate, Apple, 70 Brix | 7.500 |
| Manugel GHB, Sodium Alginate | 0.450 |
| Pectin (citrus) Type USP-L200, CP kelco | 0.850 |
| Manugel LBA, Sodium Alginate | 0.400 |
| Sodium Hexametaphosphate | 0.100 |
| EDTA, Versene CA, DOW Chem | 0.003 |
| Natural Raspberry WONF-Symrise (813750) | 1.157 |
| Flvr, Blackberry, 596447, Givaudan | 0.089 |

Results: Blood glucose response peaked 25 min after consumption of both beverages. However, the blood glucose response to the beverage containing the fiber blend was blunted (176 mg/dL vs. 192 mg/dL) as compared to the control beverage. Although blood glucose cleared more quickly in the following 25 minutes for the control, blood glucose levels after consumption of the fiber beverage cleared more quickly for the remainder of the test period. The fiber-containing beverage did not cause blood glucose levels to fall much below their starting point.
Conclusion: The alginate:pectin fiber blend in a beverage system may have utility in blood glucose control in diabetic patients.
8. Effects of Soluble Fiber and Soluble Calcium on Food Intake

The study was a within-subjects design with 30 participants completing three one week treatment periods, with a washout period of one week between treatment periods. Treatment order was counterbalanced to have five subjects randomly assigned to each of six possible treatment sequences. In each treatment period subjects consumed a test drink at breakfast and after lunch (mid-afternoon). In one treatment period, subjects consumed a placebo beverage without fiber. In two treatment periods the test drink contained a blend of soluble fibers of one of the following compositions:

| | 2.8 g Fiber | 1.0 g Fiber | Placebo |
|---|---|---|---|
| Ingredient | % | % | % |
| Water | 95.470 | 96.400 | 97.010 |
| Trisodium citrate dihydrate | 0.250 | 0.250 | 0.250 |
| LBA alginate (ISP) | 0.640 | 0.210 | 0.000 |
| GHB alginate (ISP) | 0.550 | 0.180 | 0.000 |
| USP L200 pectin (Kelco) | 0.200 | 0.066 | 0.000 |
| Apple juice concentrate | 2.300 | 2.300 | 2.300 |
| EDTA | 0.002 | 0.002 | 0.002 |
| Sucralose | 0.011 | 0.011 | 0.011 |
| Malic acid, granular | 0.200 | 0.200 | 0.200 |
| Red 40, 10% solution | 0.001 | 0.001 | 0.001 |
| Flavor | 0.380 | 0.380 | 0.380 |
| Total | 100.000 | 100.0001 | 100.000 |

The fiber drinks were consumed with a separate beverage containing calcium lactate (500 mg elemental calcium per serving). The placebo was taken with a second placebo beverage matched for flavor but without calcium lactate. The test drink containing calcium lactate or corresponding placebo had the following composition:

| | Calcium Placebo | Calcium Free Placebo |
|---|---|---|
| Ingredient | % | % |
| Water | 96.430 | 99.846 |
| Calcium lactate | 3.065 | 0.000 |
| Malic acid | 0.330 | 0.330 |
| Sucralose | 0.050 | 0.020 |
| Yellow #5, 1% solution | 0.007 | 0.007 |
| Red #40, 1% Solution | 0.0069 | 0.0069 |
| Flavor | 0.110 | 0.110 |
| Total | 100.000 | 100.000 |

Subjects in the study were premenopausal women selected without regard to racial or ethnic background. Eligible women had to be between 20 and 40 years of age, non-smokers, and overweight or obese (body mass index, or BMI, of 25-35 kg per square meter).

Test Sessions and Experimental Measurements

Test sessions occurred on the first and seventh day of each treatment period. The night before the sessions, subjects consumed an evening meal of their own choosing that was replicated the night before each test session. Test sessions began between 7:00 and 9:00 AM. Subjects first completed a short questionnaire to ensure they consumed the evening meal, and had not been ill in the previous week. Immediately before a standardized breakfast meal (choice of bagel or raisin bran cereal) they were asked to consume a fiber test beverage (consisting of two portions) within a three minute interval.

They were asked to consume the test beverage (fiber or placebo) portion first, immediately followed by the calcium beverage or placebo portion. They were then served the standard breakfast. They returned to the lab for lunch 4-5 hours later, and dinner 9-10 hours later. They were provided with a portable cooler containing the test beverage (fiber or placebo beverage, and the calcium beverage or calcium-free placebo beverage), and a bottle of water. They were instructed to consume the test drink 2½ hours after the completion of lunch. They were asked not to consume any foods during the day except the test meals provided, the test beverages, and the bottled water.

At the test sessions, lunch and dinner were provided as buffet-style meals. Subjects were also provided snacks for consumption during the evening. They were told to consume as much of the snacks as they desired. Lunch and dinner servings of each individual food were weighed to the nearest 0.1 g before and after consumption to determine caloric and macronutrient intake. Evening snacks were returned to the test site to determine food consumption.

Subjects were asked to consume 14 test drinks during each of the three week long experimental periods. On Day 1, as mentioned above, they drank one test drink before breakfast, and one 2.5 hours after lunch. Additionally, on the first test day they were provided with 10 refrigerated drink servings (each a pair of fiber drink or placebo, and the calcium beverage or calcium-free placebo beverage) to take home. They were instructed to consume one serving before breakfast, and the second 2½ hours after lunch each day on the second through sixth days. Subjects returned to the laboratory on the seventh day to repeat the procedure of the first day.

Data Analysis

Data were analyzed using the Statistical Analysis System (SAS Version 8.2, Cary, N.C.). The mixed model procedure was used to test for treatment differences, with treatment condition (low fiber, high fiber, and placebo), day (1 or 7) and the interaction of condition and day entered into the statistical models. The effects of treatment session was also tested as a covariate and kept in the final model when found to be significant. The endpoint measurements included the total daily energy and macronutrient content of foods consumed, as well as at each individual meal (breakfast, lunch, dinner, and evening snack).

Consumption of both the fiber containing beverages (1 g and 2.8 g per serving) resulted in a trend toward reduction in total calorie intake measured over the 24 hour period beginning with the morning beverage.

Effect of Fiber Beverages on Total Calorie

| | Mean | | |
|---|---|---|---|
| Condition | Kcal Intake | Standard Error | P value vs. placebo |
| Placebo | 2634 | 109 | 0.17 |
| 1 g fiber beverage | 2512 | 110 | 0.17 |
| 2.8 g fiber beverage | 2510 | 109 | |

Consumption of both the fiber containing beverages (1 g and 2.8 g per serving) resulted in a significant decrease in food consumption at dinner, as shown below.

Effect of Fiber Beverages on Caloric Intake at Dinner

| | Mean | | |
|---|---|---|---|
| Condition | Kcal Intake | Standard Error | P value vs. placebo |
| Placebo | 765 | 37 | |
| 1 g fiber beverage | 689 | 37 | 0.039 |
| 2.8 g fiber beverage | 678 | 37 | 0.016 |

The 1 g fiber beverage reduced dinner food intake by an average of 76 kcal, and the 2.8 g beverage provided a reduction of 87 kcal. The P values, determined by a post-hoc Tukey's analysis, indicated that these results were statistically significant (p<0.05).

Further analysis of the nutrient composition of the individual foods consumed indicated that the consumption of the fiber beverages was associated with a significant reduction in the intake of carbohydrates at dinner, as shown below.

Effect of Fiber Beverages on Carbohydrate Caloric Intake at Dinner

| | Mean Carbohydrate | | P value |
|---|---|---|---|
| Condition | Kcal Intake | Standard Error | vs. placebo |
| Placebo | 379 | 21 | |
| 1 g fiber beverage | 329 | 21 | 0.007 |
| 2.8 g fiber beverage | 324 | 21 | 0.003 |

The 1 g beverage reduced carbohydrate intake at dinner by 50 kcal, and the 2.8 g beverage provided a 55 kcal reduction. The reduction in carbohydrate intake at both levels was statistically significant (p<0.01).

The fiber beverages also reduced total daily food intake, as shown below.

Effects of Fiber Beverages on Daily Caloric Intake

| | Mean | | |
|---|---|---|---|
| Condition | Kcal Intake | Standard Error | P value vs. placebo |
| Placebo | 1353 | 64 | |
| 1 g fiber beverage | 1261 | 64 | 0.026 |
| 2.8 g fiber beverage | 1264 | 64 | 0.033 |

The 1 g fiber beverage reduced overall food intake on the test day by an average of 92 kcal, and the 2.8 g beverage provided a reduction of 89 kcal. The P values, determined by a post-hoc Tukey's analysis, indicated that these results were statistically significant (p<0.05). These results indicated the absence of compensatory eating that could have occurred in response to the reduced dinner caloric intake.

A number of embodiments of the invention have been described. Nevertheless, it will be understood that various modifications may be made without departing from the spirit and scope of the invention. Accordingly, other embodiments are within the scope of the following claims.

1. An ingestible composition comprising an extruded food product having a bulk density in the range of about 0.08 to about 0.25 gram/cubic centimeter, wherein said extruded food product comprises two or more anionic soluble fibers, and wherein said ingestible composition further comprises a source of one or more monovalent cations.

2. The ingestible composition of claim 1, wherein said two or more anionic soluble fibers comprise alginate and pectin.

3. The ingestible composition of claim 2, wherein said alginate comprises an intermediate molecular weight distribution range form of alginate and a low molecular weight distribution range form of alginate.

4. The ingestible composition of claim 2, wherein a ratio of total alginate to total pectin can be from about 8:1 to about 1:8.

5. The ingestible composition of claim 4, wherein said ratio of total alginate to total pectin is about 7:1.

6. The ingestible composition of claim 4, wherein said ratio of total alginate to total pectin is about 6.15:1.

7. The ingestible composition of claim 3, wherein a ratio of said intermediate molecular weight alginate to said low molecular weight alginate is about 0.65:1 to about 2:1.

8. The ingestible composition of claim 7, wherein said ratio of said intermediate molecular weight alginate to said low molecular weight alginate is about 0.8:1 to about 0.9:1.

9. The ingestible composition of claim 1, wherein said extruded food product is from about 20% to about 48% by weight of said ingestible composition.

10. The ingestible composition of claim 1, wherein said extruded food product is from about 80% to about 100% by weight of said ingestible composition.

11. The ingestible composition of claim 9, wherein said extruded food product comprises from about 22% to about 50% by weight of said two or more anionic soluble fibers.

12. (canceled)

13. The ingestible composition of claim 1, wherein said monovalent cation source is a monovalent cation salt selected from the group consisting of citrate, tartrate, succinate, fumarate, adipate, malate, lactate, gluconate, phosphate, carbonate, sulfate, chloride, and acetate.

14. The ingestible composition of claim 1, wherein said source of one or more monovalent cations is a protected source of one or more monovalent cations selected from the group consisting of:
a) a monovalent cation-alginate microparticle;
b) a monovalent cation-containing liposome; and
c) a monovalent cation-containing carbohydrate glass.

15. The ingestible composition of claim 1, wherein a ratio of said one or more monovalent cations to said two or more anionic soluble fibers in said ingestible composition ranges from about 1:20 to about 1:7.

16. The ingestible composition of claim 1, wherein said ingestible composition further comprises an edible frosting, coating, drizzle, chip, chunk, swirl, or interior layer.

17. The ingestible composition of claim 16, wherein said edible frosting, coating, drizzle, chip, chunk, swirl, or interior layer comprises a source of one or more monovalent cations.

18. A liquid ingestible composition comprising an alginate anionic soluble fiber and a pectin anionic soluble fiber, and wherein said ingestible composition further comprises a source of one or more monovalent cations.

19. The liquid ingestible composition of claim 18, wherein said pectin anionic soluble fiber is a high-methoxy pectin.

20. The liquid ingestible composition of claim 18, wherein said alginate anionic soluble fiber has a ratio of guluronic:mannuronic acid units of 1.2:1 to 1.8:1.

21. The liquid ingestible composition of claim 18, wherein said alginate anionic soluble fiber comprises two or more alginate forms.

22. The liquid ingestible composition of claim 18, wherein said alginate is selected from the group consisting of Manugel GHB alginate, Manugel LBA alginate, and Manugel DPB alginate.

23. The liquid ingestible composition of claim 21, wherein said two or more alginate forms include an intermediate molecular weight distribution range alginate form and a low molecular weight distribution range alginate form.

24. The liquid ingestible composition of claim 23, wherein said intermediate molecular weight alginate form is Manugel GHB alginate.

25. The liquid ingestible composition of claim 23, wherein said low molecular weight alginate form is Manugel LBA alginate.

26. The liquid ingestible composition of claim 18, wherein said composition has a pH ranging from about 3.9 to about 4.5.

27. The liquid ingestible composition of claim 18, wherein said alginate and said pectin comprise about 1.0% to about 2.2% of said liquid ingestible composition by weight.

28. The liquid ingestible composition of claim 27, wherein said alginate and said pectin comprise about 1.25% to about 1.8% of said ingestible composition by weight.

29. The liquid ingestible composition of claim 18, wherein said composition has a viscosity of from about 15 to 200 cPs.

30. The liquid ingestible composition of claim 18, wherein a ratio of said alginate to said pectin ranges from about 8:1 to about 1:8.

31. The liquid ingestible composition of claim 18, wherein said liquid ingestible composition exhibits a gel strength of about 20 to about 250 grams Force as measured in a gel strength assay.

32. The liquid ingestible composition of claim 18, further comprising a cation sequestrant.

33. The liquid ingestible composition of claim 32, wherein said cation sequestrant is selected from the group consisting of EDTA salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate.

34. An article of manufacture comprising:
a) the liquid ingestible composition of claim 18; and
b) a source of one or more monovalent cations.

35. The article of claim 34, wherein said source of one or more monovalent cations is a liquid.

36. The article of claim 34, wherein said liquid ingestible composition and said source of one or more monovalent cations are in separate containers.

37. A fiber blend comprising an alginate anionic soluble fiber and a pectin anionic soluble fiber, and wherein said fiber blend further comprises a source of one or more monovalent cations.

38. The fiber blend of claim 38, wherein said alginate anionic soluble fiber includes a intermediate molecule weight distribution range alginate form and a low molecular weight distribution range alginate form.

39. The fiber blend of claim 38, wherein a ratio of total alginate to total pectin in the fiber blend can be from about 8:1 to about 5:1.

40. The fiber blend of claim 40, wherein said ratio is about 7:1.

41. The fiber blend of claim 40, wherein said ratio is about 6.15:1.

42. The fiber blend of claim 39, wherein a ratio of said intermediate molecular weight alginate to said low molecular weight alginate is about 0.65:1 to about 2:1.

43. The fiber blend of claim 43, wherein said ratio of said intermediate molecular weight alginate to said low molecular weight alginate is about 0.8:1 to about 0.9:1.

44. An extruded food product having a bulk density in the range from about 0.08 to about 0.25 gram/cubic centimeter, wherein said extruded food product comprises two or more anionic soluble fibers, and wherein said extruded food product further comprises a source of one or more monovalent cations.

45. The extruded food product of claim 45, wherein said two or more anionic soluble fibers comprise alginate and pectin.

46. The extruded food product of claim 46, wherein said alginate comprises an intermediate molecular weight distribution range form of alginate and a low molecular weight distribution range form of alginate.

47. The extruded food product of claim 46, wherein a ratio of total alginate to total pectin can be from about 8:1 to about 5:1.

48. The extruded food product of claim 48, wherein said ratio of total alginate to total pectin is about 7:1.

49. The extruded food product of claim 48, wherein said ratio of total alginate to total pectin is about 6.15:1.

50. The extruded food product of claim 47, wherein a ratio of said intermediate molecular weight alginate to said low molecular weight alginate is about 0.65:1 to about 2:1.

51. The extruded food product of claim 47, wherein said ratio of said intermediate molecular weight alginate to said low molecular weight alginate is about 0.8:1 to about 0.9:1.

52. The extruded food product of claim 45, wherein said extruded food product comprises from about 22% to about 50% by weight of said two or more anionic soluble fibers.

53. A method of making the extruded food product of claim 45, comprising extruding a composition comprising said two or more anionic soluble fibers through a die under heat and high-pressure.

54. A method of delaying the reaction of a monovalent cation with one or more anionic soluble fibers in an ingestible composition until after ingestion of said ingestible composition, comprising preparing a protected source of said monovalent cation and adding said protected source to an ingestible composition.

55. A method of delaying the hydration of one or more anionic soluble fibers in an ingestible composition until after ingestion, said method comprising co-processing the one or more anionic soluble fibers with one or more ingredients selected from protein sources, starch, cereal ingredients, maltodextrin, inulin, cocoa solids, corn fiber, vegetable oils, and sucralose, wherein said ingestible composition comprises a source of one or more monovalent cations.

56. The method of claim 56, wherein said one or more ingredients comprises one or more cereal ingredients selected from rice, corn, wheat, sorghum, oat, flax and barley grains, flours, and meals.

57. The method of claim 56, wherein said one or more ingredients comprises one or more protein sources selected from rice, milk, egg, wheat, whey, soy, and gluten protein.

58. The method of claim 56, wherein said one or more ingredients comprises a protein concentrate or isolate.

59. (canceled)

60. The liquid ingestible composition of claim 18, wherein said monovalent cation is selected from Na+, Li+, K+, and Rb+.

61. The liquid ingestible composition of claim 18, wherein said source of one or more monovalent cations is a protected source of one or more monovalent cations selected from the group consisting of:
a) a monovalent cation-alginate microparticle;
b) a monovalent cation-containing liposome; and
c) a monovalent cation-containing carbohydrate glass.
